(19) United States
(12) Patent Application Publication
(10) Pub. No.: US 2020/0316002 A1
(43) Pub. Date: Oct. 8, 2020

(54) PREVENTION OF URINARY TRACT DEVICE ENCRUSTATION

(71) Applicant: Devicare SL, Cerdanyola del Vallès (ES)

(72) Inventor: Jordi CUÑE, Rubí (ES)

(73) Assignee: Devicare SL, Cerdanyola del Vallès (ES)

(21) Appl. No.: 16/838,760

(22) Filed: Apr. 2, 2020

Related U.S. Application Data
(60) Provisional application No. 62/828,883, filed on Apr. 3, 2019.

(51) Int. Cl. A61K 31/198; A61K 31/6615; A61K 31/704; A61K 31/194; A61K 33/08; A61K 36/88; A61K 36/87; A61K 33/30; A61K 31/191; A61K 31/07; A23L 33/105; A23L 33/125; A23L 33/155; A23L 33/165; A23L 33/175

(52) U.S. Cl. CPC A61K 31/198; A23L 33/175; A61K 31/704; A61K 31/194; A61K 33/08; A61K 36/88; A61K 36/87; A61K 33/30; A61K 31/191; A61K 31/07; A23L 33/105; A23L 33/125; A23L 33/155; A23L 33/165; A61K 31/6615

(57) ABSTRACT

The present disclosure provides compositions and methods to prevent or reduce the encrustation of medical devices inserted in the urinary tract, such as a urinary stents (e.g., JJ stents). The reduction or prevention can result in an improvement in the quality of life of the patient by, e.g., reducing pain and/or tissue damage caused during the removal of an encrusted device.

### FIELD OF THE DISCLOSURE

The present disclosure relates to compositions and methods to prevent or reduce the encrustation of medical devices inserted in the urinary tract.

### BACKGROUND

Double-J (JJ) ureteral stents (Finney, J. Urol. 1978, 120:678-81) are one of the most common indwelling ureteral devices used for treatment of obstructive uropathy, postoperative of ureteropyelic stenosis and renal transplantation (González-Ramirez et al., J. Rev Mex Urol. 2009; 69(1):7-12; Saltzman B., Urol Clin North Am. 1988; 15:481-491). Their effectiveness for renal collecting system drainage has been proven (Türk et al., Eur Urol. 2016; 69(3):468-74) and their characteristic design, with both renal and vesical J-shaped curl ends, prevents stent migration (Finney, J. Urol. 1978, 120:678-81). However, double J ureteral stents have also been related to patient discomfort, pain, urinary tract infection, and encrustation (Stickler, J Intern Med. 2014; 276:120-9; Beysens & Tailly, Asian J Urol. 2018; 5:274-86).

The management of the forgotten, encrusted, or calcified ureteral stent can represent one of the most difficult and challenging surgical conditions for the practicing urologist. The challenges in managing these patients result from the potential need for multiple surgical interventions (Acosta-Miranda et al., J Endourol. 2009; 23(3):409-15). A prolonged indwell time of stents, as well as a history of nephrolithiasis and urinary infections may on many occasions result in calcification and encrustation of ureteral stents, and will lead to the use of endourology techniques, extracorporeal lithotripsy, or open surgery to resolve these conditions (Arrabal Polo et al., Arch. Esp. Urol. 2010; 63(10):873-6).

Biofilm-formation is a multistep process that seems to be involved in the occurrence of encrustations (Sighinolfi et al., Urology. 2015; 86: 703-6; Burr & Nuseibeh, Spinal Cord. 1997; 35:521-5) which may lead to stent obstruction or stent removal complications (Beysens & Tailly, Asian J Urol. 2018; 5:274-86). Shortly after the stent insertion, different organic molecules adhere to its surface forming a multi-layered complex structure. Until now, the presence of bacteria attached to the stent surface was considered indispensable and directly associated with the formation of struvite and hydroxyapatite crystals (Stickler, J Intern Med. 2014; 276:120-9; Zumstein et al., Swiss Med Wkly. 2017; 3:147), but recent studies have demonstrated that the presence of bacteria are not compulsory and organic film, together with urine pH, might play a bigger role in Ca and Mg phosphate precipitates forming struvite and hydroxyapatite crystals, which result in stent encrustation (Grases F, 2019). In addition to this mechanism, other factors such as stent material, stent coating, urine composition and flow dynamics are also involved (Mosayyebi et al., Current Urology Reports. 2018; 19:35). In this regard, urinary pH plays an important role and several studies have shown that abnormal urine pH values are found in blocker patients (those in which stent obstruction is observed) compared to non-blocker ones (Burr et al., Spinal Cord. 1997; 35:521-5; Hedelin et al., Br J Urol. 1991; 67(5):527-32; Kohler-Ockmore & Feneley, Br J Urol. 1996; 77(3):347-51).

Accordingly, there is a need for the development of new methods to reduce or prevent encrustation on the surface to urinary stents (e.g., JJ stents) and other medical devices inserted in the urinary tract that may be susceptible to encrustation.

### BRIEF SUMMARY

The present disclosure provides methods to prevent encrustation on urinary tract device in a subject in need thereof comprising administering to the subject:
at least one dose of an oral composition that decreases urine pH, e.g., if the subject's urine pH is above a pH threshold or pH range (e.g., as measured with a POC pH meter disclosed herein);
(ii) at least one dose of an oral composition that increases urine pH, e.g., if the subject's urine pH is below a pH threshold or pH range (e.g., as measured with a POC pH meter disclosed herein);
(iii) at least one dose of an oral composition that maintains urine pH, e.g., if the subject's urine pH within a pH range (e.g., as measured with a POC pH meter disclosed herein); or,
(iv) a combination thereof;
   wherein the administration of at least one composition to the subject reduces encrustation on the surface of the urinary tract device.

The present disclosure also provides methods to facilitate the removal of a urinary tract device in a subject in need thereof comprising administering to the subject:
(i) at least one dose of an oral composition that decreases urine pH, e.g., if the subject's urine pH is above a pH threshold or pH range (e.g., as measured with a POC pH meter disclosed herein);
(ii) at least one dose of an oral composition that increases urine pH, e.g., if the subject's urine pH is below a pH threshold or pH range (e.g., as measured with a POC pH meter disclosed herein);
(iii) at least one dose of an oral composition that maintains urine pH, e.g., if the subject's urine pH within a pH range (e.g., as measured with a POC pH meter disclosed herein); or,
(iv) a combination thereof;
   wherein the administration of at least one composition to the subject reduces encrustation on the surface of the urinary tract device, and the reduced encrustation facilitates the removal of the urinary tract device.

In some aspects, the oral composition that decreases urine pH comprises, e.g., a urinary acidifying agent and a calcium phosphate crystallization inhibitor. In some aspects, the urinary acidifying agent is an amino acid or a combination thereof. In some aspects, the amino acid is L-methionine. In some aspects, the calcium phosphate crystallization inhibitor comprises phytic acid, a salt thereof, or a combination thereof.

In some aspects, the oral composition that increases urine pH comprises, e.g., a urinary basifying agent and a uric acid crystallization inhibitor. In some aspects, the urinary basifying agent comprises citric acid, salts thereof, or combinations thereof. In some aspects, the uric acid crystallization inhibitor comprises a saponin.

In some aspects, the oral composition that maintains urine pH comprises, e.g., (a) a calcium phosphate crystallization inhibitor, and (b) a magnesium salt, magnesium hydroxide, magnesium oxide, or a combination thereof. In some aspects, the calcium phosphate crystallization inhibitor comprises, e.g., phytic acid, a salt thereof, or a combination thereof. In some aspects, the oral composition that maintains urine pH comprises, e.g., magnesium oxide. In some aspects, the oral composition that maintains urine pH further comprises at least one polyphenol.

In some aspects, the urinary tract device is a stent. In some aspects, the stent is a JJ stent. In some aspects, the administration of the compositions to the subject decreases encrustation on the surface of the urinary tract device by at least 1-fold, at least 2-fold, at least 3-fold, at least 4-fold, at least 5-fold, at least 6-fold, at least 7-fold, at least 8-fold, at least 9-fold, or at least 10-fold compared to the encrustation observed in control subjects. In some aspects, the administration of the compositions to the subject reduced the risk of encrustation on the surface of the urinary tract device by at least 1-fold, at least 2-fold, at least 3-fold, at least 4-fold, at least 5-fold, at least 6-fold, at least 7-fold, at least 8-fold, at least 9-fold, or at least 10-fold compared to the risk of encrustation observed in control subjects.

In some aspects, the methods disclosed herein further comprise the measurement of urine pH by the subject using a self-diagnostic pH measuring device.

In some aspects, the oral composition that decreases urine pH comprises L-methionine, rice husk extract (rice bran extract) enriched in phytic acid magnesium and calcium salts, zinc gluconate, and vitamin A. In some aspects, the oral composition that increases urine pH comprises potassium citrate, magnesium citrate, a saponin, zinc gluconate, and vitamin A. In some aspects, the composition that maintains urine pH comprises rice husk extract enriched in phytic acid magnesium and calcium salts, magnesium oxide, grape seed extract enriched in polyphenols, zinc gluconate, and vitamin A.

### BRIEF DESCRIPTION OF THE DRAWINGS/FIGURES

FIG. 1A-1C show the surface of a stent exclusively covered by an organic matter layer (encrustation classified as 1) (FIG. 1A); surface of a stent with spheroidal aggregates of calcium oxalate monohydrate crystals embedded in an organic matter layer (FIG. 1B); Surface of a stent totally covered by a layer of calcium oxalate monohydrate crystals developed on a layer of organic matter (FIG. 1C).

FIG. 2 shows the surface of a stent covered by an organic matter layer in which colonies of bacteria have developed (encrustation classified as 2).

FIG. 3A-3C show the surface of a stent covered by encrustations classified as 2. FIG. 3A, FIG. 3B: Hydroxyapatite deposits. FIG. 3C, FIG. 3D: calcium oxalate dihydrate+calcium oxalate monohydrate deposits.

FIG. 4A and FIG. 4B show the surface of a stent covered by important deposits of Brushite+hydroxyapatite, classified as 3.

FIG. 5A and FIG. 5B show the surface of a stent covered by ammonium magnesium phosphate+hydroxyapatite deposits, classified as 3.

FIG. 6A and FIG. 6B show the surface of a stent covered by dihydrate uric acid deposits, classified as 3.

FIG. 7 shows study flow diagram.

FIG. 8 shows encrustation measurement of Double J ureteral stent.

FIG. 9 shows characteristics of the study population.

FIG. 10 shows the outcome measures of the study between study groups.

FIG. 11 shows multivariate model of Double J ureteral stent encrustation.

FIG. 12 shows the distribution of encrustation levels at the kidney end and at the bladder end in the total of patients at the end of the study.

FIG. 13A and FIG. 13B show global calcification comparison (value=4) between groups for the totality of Double J stent ends (n=198). FIG. 13A: Graphic representation of global calcification according to group. FIG. 13B: Table of results.

FIG. 14A and FIG. 14B show binary logistic regression: global calcification (value=4) for the totality of Double J stent ends (n=198). FIG. 14A: Graphic representation.

FIG. 14B: Table of results. Exp(B)=Magnitude of the effect (protective or inducer of encrustation).

FIG. 15A and FIG. 5B show deposit levels comparison between groups for all the compiled Double J stent ends (n=198). FIG. 15A: Graphic representation. FIG. 15B: Table of results.

FIG. 16A and FIG. 16B show general linear model: deposit levels for the totality of Double J stent ends (n=198). FIG. 16A: Graphic representation. FIG. 16B: Table of results. Partial Eta²=Magnitude of the effect (protective or inducer of encrustation).

FIG. 17A-FIG. 17C show the relation between encrustation and days of stent implantation in the experimental and placebo groups. FIG. 17A: Table of results. FIG. 17B, FIG. 17C: Graphs and projections (FIG. 17B: Placebo, FIG. 17C: Experimental).

FIG. 18A and FIG. 18B show encrustation/calcification comparison between groups: Deposit levels at kidney end. FIG. 18A: Graphic representation. FIG. 18B: Table of results.

FIG. 19A and FIG. 19B show encrustation/calcification comparison between groups: Deposit levels at bladder end. FIG. 19A: Graphic representation. FIG. 19B: Table of results.

FIG. 20A and FIG. 20B show encrustation/calcification comparison between groups: Sum of stent ends. FIG. 20A: Graphic representation. FIG. 20B: Table of results.

FIG. 21A and FIG. 21B show encrustation/calcification comparison between groups: Maximum deposit values at stent ends. FIG. 21A: Graphic representation. *Maximum encrustation. FIG. 21B: Table of results.

FIG. 22A and FIG. 22B show analytical pH reduction baseline vs day 21. Panel FIG. 22A: Graphic representation. FIG. 22B: Table of results.

FIG. 23A and FIG. 23B show mean pH values during stent implantation and the mean pH values for days 1-3. FIG. 23A: Graphic representation. FIG. 23B: Table of results.

FIG. 24A and FIG. 24B shows the difference in the regression slope for the pH values along the stent implantation days. FIG. 24A: Graphic representation. FIG. 24B: Table of results.

FIG. 25A and FIG. 25B show the cases of impossibility of stent removal in the first trial.

FIG. 26A and FIG. 26B shows duration time for the stent removal (FIG. 26A) and adjustment of results to factors as previous implantation and symptoms associated with the presence of the stent (FIG. 26B).

### DETAILED DESCRIPTION

The present disclosure provides methods to prevent and/or reduce encrustation on urinary tract devices (e.g., stents such as JJ stents, also known as Double J stents or Double Pigtail stents) in a subject in need thereof based on maintaining urine pH below or above a predetermined threshold pH value (as measured, e.g., with a POC pH meter disclosed herein) or within a predetermined pH range to prevent or reduce pH-related encrustation (e.g., due to hydroxyapatite or oxalate crystallization) on the surface of the urinary tract device.

Maintenance of urine pH within a desired pH range, or above or below a pH threshold that would trigger encrustation, can be accomplished by administering to the subject (or self-administering) one or more oral compositions that raise, lower, or maintain the urine pH at the desired level. For example, if the subject's urine pH is above a pH threshold or pH range that may trigger encrustation or worsen encrustation (e.g., increase rate and/or magnitude of encrustation), the subject can ingest at least one dose of an oral composition that decreases urine pH. If the subject's urine pH is below a pH threshold or pH range that may trigger encrustation or worsen encrustation, the subject can ingest at least one dose of an oral composition that increases urine pH. And if the subject's urine pH is within a desired pH range or close to a desired urine pH value, the subject can ingest at least one dose of an oral composition that maintains urine pH.

In some aspects, this process can be conducted dynamically in response to measured variations in urine pH (e.g., as measured with a POC pH meter disclosed herein). Thus, for example, if the urine pH is too low, the subject can ingest an oral composition disclosed herein that increases urine pH, but if at some point the urine pH becomes too high, the subject may ingest an oral composition disclosed herein that lowers urine pH, so the urine pH was fall below a predetermined threshold pH known to trigger encrustation, or fall within a urine pH range in which encrustation does not occur or at least is reduced. Once the urine pH is below or above the predetermined urine pH threshold or within the urine pH range in which encrustation does not occur or is reduced, the subject can use an oral composition disclosed herein that maintains the urine pH at the desired level as, e.g., as a maintenance treatment.

To practice the methods disclosed herein, urine pH measurements can be performed using a self-monitoring device (e.g., a portable device) with sufficient sensitivity (e.g., 0.1 pH units) in order to provide quick and reproducible pH measurements across a pH range encompassing the predetermined threshold pH values and/or the desired pH range, for example, a LIT-CONTROL DEVICARE® point-of-care pH meter described, e.g., at www.devicare.com/en/devicare-obtiene-la-patente-en-europa-y-estados-unidos-de-su-tecnologia-sensorica/.

Before the present disclosure is described in greater detail, it is to be understood that this invention is not limited to the particular compositions or process steps described, as such can, of course, vary. As will be apparent to those of skill in the art upon reading this disclosure, each of the individual aspects described and illustrated herein has discrete components and features which can be readily separated from or combined with the features of any of the other several aspects without departing from the scope or spirit of the present invention. Any recited method can be carried out in the order of events recited or in any other order which is logically possible.

The headings provided herein are not limitations of the various aspects of the disclosure, which can be defined by reference to the specification as a whole. It is also to be understood that the terminology used herein is for the purpose of describing particular aspects only, and is not intended to be limiting, since the scope of the present disclosure will be limited only by the appended claims.

Accordingly, the terms defined immediately below are more fully defined by reference to the specification in its entirety.

### I. Definitions

In order that the present description can be more readily understood, certain terms are first defined. Additional definitions are set forth throughout the detailed description.

It is to be noted that the term “a” or “an” entity refers to one or more of that entity; for example, “a pH measurement,” is understood to represent one or more pH measurement. As such, the terms “a” (or “an”), “one or more,” and “at least one” can be used interchangeably herein. It is further noted that the claims can be drafted to exclude any optional element. As such, this statement is intended to serve as antecedent basis for use of such exclusive terminology as “solely,” “only” and the like in connection with the recitation of claim elements, or use of a negative limitation.

Furthermore, “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

It is understood that wherever aspects are described herein with the language “comprising,” otherwise analogous aspects described in terms of “consisting of” and/or “consisting essentially of” are also provided.

Unless defined otherwise, all technical and scientific terms used herein have the same meaning as commonly understood by one of ordinary skill in the art to which this disclosure is related. For example, the Concise Dictionary of Biomedicine and Molecular Biology, Juo, Pei-Show, 2nd ed., 2002, CRC Press; The Dictionary of Cell and Molecular Biology, 3rd ed., 1999, Academic Press; and the Oxford Dictionary Of Biochemistry And Molecular Biology, Revised, 2000, Oxford University Press, provide one of skill with a general dictionary of many of the terms used in this disclosure.

Units, prefixes, and symbols are denoted in their Systéme International de Unites (SI) accepted form. Numeric ranges are inclusive of the numbers defining the range. Where a range of values is recited, it is to be understood that each intervening integer value, and each fraction thereof, between the recited upper and lower limits of that range is also specifically disclosed, along with each subrange between such values. The upper and lower limits of any range can independently be included in or excluded from the range, and each range where either, neither or both limits are included is also encompassed within the disclosure. Thus, ranges recited herein are understood to be shorthand for all of the values within the range, inclusive of the recited endpoints. For example, a range of 1 to 10 is understood to include any number, combination of numbers, or sub-range from the group consisting of 1, 2, 3, 4, 5, 6, 7, 8, 9, and 10 (e.g., 1 to 2, 2 to 5, 5 to 7, 4.5 to 6.7, 5.5 to 6.5, etc.).

Where a value is explicitly recited, it is to be understood that values which are about the same quantity or amount as the recited value are also within the scope of the disclosure. Where a combination is disclosed, each subcombination of the elements of that combination is also specifically disclosed and is within the scope of the disclosure. Conversely, where different elements or groups of elements are individually disclosed, combinations thereof are also disclosed. Where any element of a disclosure is disclosed as having a plurality of alternatives, examples of that disclosure in which each alternative is excluded singly or in any combination with the other alternatives are also hereby disclosed; more than one element of a disclosure can have such exclusions, and all combinations of elements having such exclusions are hereby disclosed.

About: The term “about” is used herein to mean approximately, roughly, around, or in the regions of. When the term “about” is used in conjunction with a numerical range, it modifies that range by extending the boundaries above and below the numerical values set forth. In general, the term “about” can modify a numerical value above and below the stated value by a variance of, e.g., 10 percent, up or down (higher or lower).

Administer: The terms “administration,” “administering,” and grammatical variants thereof refer to introducing a composition (e.g., a composition of the present disclosure) into a subject via a pharmaceutically or nutraceutically acceptable route, e.g., orally. Administration includes self-administration and the administration by another (e.g., a clinician). A suitable route of administration, e.g., oral administration, allows the composition to perform its intended function. In some aspects, the terms administration and treatment can be used interchangeably.

Approximately: As used herein, the term “approximately,” as applied to one or more values of interest, refers to a value that is similar to a stated reference value. In certain aspects, the term “approximately” refers to a range of values that fall within 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

Composition of the present disclosure: The term “composition of the present disclosure” and grammatical variants thereof refers collectively to the composition that decreases urine pH, composition that increases urine pH, and composition that maintains urine pH, described below, and combinations thereof.

Composition that decreases urine pH: As used herein, the phrase “composition that decreases urine pH” refers to a composition or combination thereof suitable for oral administration that when ingested by a subject acidifies (i.e., decreases or lowers) the subject's urine pH.

Composition that increases urine pH: As used herein, the phrase “composition that increases urine pH” refers to a composition or combination thereof suitable for oral administration that when ingested by a subject basifies (i.e., increases or elevates) the subject's urine pH.

Composition that maintains urine pH: As used herein, the phrase “composition that maintains urine pH” refers to a composition or combination thereof suitable for oral administration that when ingested by a subject maintains the subject's urine pH within a desired pH range (i.e., stabilizes the pH, keeps a constant or substantially constant pH, or reduces fluctuations in urine pH).

Encrustation: As used herein, the term “encrustation” refers to the formation of deposits, e.g., deposits of calcified material, on a medical device inserted in the urinary tract of a subject, e.g., a urinary catheter or stent such as a JJ stent. The term encrustation refers both to the process of deposition on the medical device and to the deposit accumulated on the medical device (i.e., encrusted material, encrusted deposit). In some aspects, the terms encrustation and calcification are used interchangeably.

Encrustation can refer, e.g., to the formation of deposits of oxalates (e.g., calcium oxalate), calcium phosphates (e.g., brushite and/or hydroxyapatite), uric acid, magnesium ammonium sulfate, struvite (magnesium ammonium phosphate), or combinations thereof.

In some aspects, encrustation can be determined by visual examination or observation of the urinary track device after extraction or in situ via endoscopy. In other aspects, encrustation can be measured for example using scanning electron microscopy, micro-analysis by dispersive energy of X rays, or by inductively coupled plasma atomic emission spectroscopy (ICP-AES). In some aspects, the scale of encrustation can be determined according to a scale, e.g., 0 (without encrustation), 1 (sporadic calcifications/encrustations less than 1-2 mm²), 2 (calcifications/encrustations of wide area greater than 1-2 mm²), or 3 (complete blockage/obstruction/occlusion). In other aspects, a 5 point score (0=none, 1=biofilm, 2=few crystals, 3=many crystals, 4=global calcification) scale can be used to quantify encrustation. In some aspects, the risk of encrustation can be estimated or calculated by applying a blockage risk factor analysis (Clifford, 200, Community Nurse 6(4):35-6) or other methods known in the art.

Excipient: The terms “excipient” and “carrier” are used interchangeably and refer to an inert substance added to a pharmaceutical or nutraceutical composition to further facilitate administration of a compound. In addition to active ingredients that can modify the pH and/or affect crystallization of ions present in urine, the compositions of the present disclosure can contain one or more excipients.

Extract: As used herein the term “extract” as applied to a “plant extract” refers to a concentrate or isolated fraction that can be obtained from any part of a plant, e.g., seeds, leaves, fruit, stems, bark, roots, or parts thereof (e.g., seed husks, fruit skins, etc.), or combinations thereof, and contains an appreciable amount by weight of the total weight of the compound useful to practice the disclosed methods, for example phytic acid and/or salts thereof, polyphenols, or saponins.

In one specific aspect, the plant extract is, e.g., a rice husk extract (also known as a rice bran extract) enriched in phytic acid magnesium and calcium salts. In another, aspects, the plant extract is, e.g., a plant extract enriched in saponins. In yet another aspects, the plant extract is a grape seed extract enriched in polyphenols.

Isolated: As used herein, the terms “isolated,” “purified,” “extracted,” and grammatical variants thereof are used interchangeably and refer to a compound, e.g., saponin, phytate, or a polyphenol, used in the preparation of a composition of the present disclosure that has undergone one or more processes of purification.

In some aspects, isolating or purifying as used herein is the process of removing, partially removing (e.g., impurities) from a compound used to manufacture a composition of the present disclosure. In some aspects, an isolated compound has no detectable undesired activity or, alternatively, the level or amount of the undesired activity is at or below an acceptable level or amount.

In some aspects, the isolated compound or enriched extract (e.g., saponin, phytate, or a polyphenol) is enriched as compared to the starting material from which the composition is obtained. This enrichment can be by at least about 10%, at least about 20%, at least about 30%, at least about 40%, at least about 50%, at least about 60%, at least about 70%, at least about 80%, at least about 90%, at least about 95%, at least about 96%, at least about 97%, at least about 98%, at least about 99%, at least about 99.9%, at least about 99.99%, at least about 99.999%, at least about 99.9999%, or greater than 99.9999% as compared to the starting material. In some aspects, the isolated compound or enriched extract (e.g., saponin, phytate, or a polyphenol) is substantially free of residual (i.e., undesired) biological products.

In some aspects, the isolated compound or enriched extract (e.g., saponin, phytate, or a polyphenol) is 100% free, at least about 99% free, at least about 98% free, at least about 97% free, at least about 96% free, at least about 95% free, at least about 94% free, at least about 93% free, at least about 92% free, at least about 91% free, or at least about 90% free of any contaminating or residual biological matter. Residual biological products can include, e.g., abiotic materials (including chemicals) or unwanted nucleic acids, proteins, lipids, carbohydrates, or metabolites.

JJ Stent: The terms “JJ stent,” “double J stent,” and “double pigtail stent” are used interchangeably and refer to a specially designed hollow tube, made of a flexible plastic material that is placed in the ureter and drains urine from the kidney to the bladder and it expect to stay in place temporarily or permanently. The ureter is the natural tube that transmits urine from the kidney to the bladder. The length of the stents used in adult patients varies between 24 to 30 cm. Because the stent coils like a J in the bladder and like an inverted J in the kidney, it is referred to as a JJ stent.

Pharmaceutical composition: As used herein, the term “pharmaceutical composition” refers to one or more of the composition described herein mixed or intermingled with, or suspended in one or more other chemical components, such as pharmaceutically-acceptable carriers and excipients. One purpose of a pharmaceutical composition is to facilitate administration of compounds that regulate urine pH to a subject. In some aspects of the present disclosure, the terms “pharmaceutical composition” and “nutraceutical composition” can be used interchangeably. In some aspects, a composition of the present disclosure is a pharmaceutical composition.

Phytate: As used herein, the term “phytate” refers to phytic acid and its salts, as well as other polyphosphorylated inositol compounds known in the art (e.g., IP2, IP3, IP4 or IP5). In a strict sense, the terms “phytate,” “phytic acid,” and “phytin,” may be differentiated as follows: “phytate” refers to an anionic form of phytic acid; “phytic acid” refers to inositol hexaphosphate, a compound that occurs naturally in plants, including particularly plant leaves, and that may serve as a substrate for the enzyme phytase; and “phytin” refers to a salt of phytic acid, such as a calcium-magnesium salt of phytic acid. It is understood, accordingly, that “phytate,” “phytic acid,” and “phytin” are chemically related and interconvertible forms having a shared chemical structure. As used herein, therefore, “phytate,” “phytic acid,” and “phytin” are interchangeable terms in as much as they are highly related, similar, chemically interconvertible. Thus, where only one of the terms “phytate,” “phytic acid,” or “phytin” is used in the descriptions of the methods disclosed herein or in any documents incorporated by reference in the present disclosure, it is understood to function as a representative term.

In some aspects of the present disclosure “phytic acid” or “myo-inositol-hexaphosphate,” is understood as the molecule of the formula:

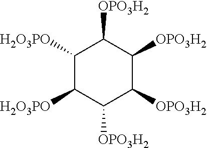

and “phytate” is the form of phytic acid with at least one less hydrogen, so that the corresponding phosphate group interact with a cation (K, Mg, Ca, Mn, Zn, or Fe). In some aspects of the present disclosure, phytate salts are sodium, potassium, calcium, magnesium, zinc, or calcium-magnesium salts, or combinations thereof.

Polyphenol: As used herein, the term “polyphenol” refers to compounds which comprise more than one phenolic group per molecule. These products have significant antioxidant properties and are also suitable for human consumption. Among the polyphenols are the following classes: flavonoids (a term often used to denote polyphenols in general, but more commonly in Europe to denote only the flavones), the flavonols, proanthocyanidins (also called procyanidols, procyanins, procyanidins and tannins) and anthocyanins. Within the general term “polyphenols” are included the dihydroxy- or tri-hydroxy benzoic acids and the phytoalexins, a typical example of which is resveratrol.

Polyphenols are found in various amounts in large numbers of natural products especially plant materials such as fruit and vegetables. In particular, polyphenols occur in grape skins and grape seeds. Grape seeds contain chiefly one class of polyphenols, the proanthocyanidins, with catechin, epicatechin and its esters as minor components. Accordingly, in some aspects, the extracts enriched in polyphenols used in compositions of the present disclosure, are obtained from grape seed.

In some aspects, the polyphenols used in the compositions of the present disclosure are obtained, e.g., from berries, tea, beer, olive oil, chocolate/cocoa, nuts, or pomegranates. Specific polyphenols that can be used to practice the methods of the present disclosure comprise, e.g., epicatechin, catechin, gallocatechin, epigallocatechin, quercetin, resveratrol, tannic acid, gallic acid, or any combinations thereof.

Prevent: As used herein, the terms “prevent,” “preventing,” and grammatical variants thereof refers, e.g., to partially or completely inhibiting encrustation of a urinary tract device; delaying onset of encrustation of a urinary tract device; reducing the amount of encrustation of a urinary tract device; or reducing the rate of encrustation of a urinary tract device. With respect to other effects resulting from the practice of the methods of the present disclosure, prevent refers, e.g., also to partially or completely inhibiting, delaying the onset, reducing the severity, reducing the rate, or reducing the amount of a undesired effect (e.g., pain or tissue damage caused by removal of an encrusted stent). In some aspects, preventing encrustation is achieved through prophylactic treatment.

The methods disclosed herein can be used also to “improve” or to “maintain” homeostasis. Thus, for example, when the methods disclosed herein prevent or reduce pain, discomfort, tissue damage, urinary tract device (e.g., stent) encrustation, occlusion or obstruction of a urinary tract device (e.g., a JJ stent), such methods simultaneously “improve” or “maintain” desired effects, for example, they improve or maintain the subject's well being, the subject's quality of life, urinary tract tissue health, or urinary track device functionality, among others.

Point of Care: The terms “Point-of-Care” or “POC” refers to devices, generally portable devices, that allow patients, physicians, and medical staff to accurately achieve real-time, lab-quality diagnostic results within second or minutes instead of hours. Point-of-care testing, e.g., home testing or bedside testing, is defined as medical diagnostic testing at or near the point of care—that is, at the time and place of patient care. This contrasts with the historical pattern in which testing was wholly or mostly confined to the medical laboratory. In some aspects of the present disclosure, POC measurement of urine pH is conducted at the patient's home or a patient's preferred location. In some aspects, POC measurements of urine pH are conducted using a portable pH meter, e.g., an electronic sensor-based portable pH meter such as a LIT-CONTROL DEVICARE® pH Meter.

Prophylactic: As used herein, “prophylactic” refers to a therapeutic or preventive course of action used to prevent, e.g., encrustation and/or delay the onset of encrustation.

Prophylaxis: As used herein, a “prophylaxis” refers to a measure taken to maintain health and prevent, e.g., encrustation and/or delay the onset of encrustation.

Saponin: The term “saponin” as used herein refers to glycosides of steroids, steroid alkaloids or triterpenes present in plants, for example, in the plant skins where they a form a waxy protective coating. Numerous saponins, as well as methods for their extraction, purification, and characterization are known in the art. See, e.g., Hostettmann et. al., British Journal of Nutrition, 88 (6): 587-605 (1995); U.S. Pat. No. 8,298,590, and Xu et al., Advances in Experimental Medicine and Biology, 404:371-82 (1996). In some aspects, the term saponin refers to, for example, triterpenic saponin, steroidal saponin, alkaloid saponin or any combination thereof depending on the chemical structure of the sapogenin. The biological and chemical activities of saponins are directly related to the number of sugar chains bound to the sapogenin, for example, saponins that have one sugar chain attached to the sapogenin are called mono-desmosidic saponins; saponins with sapogenins that have two sugar chains are called bi-desmosidic saponins. See e.g., Waller, G. R. et. al., “Saponins used in Traditional and Modern Medicine, Advances in Experimental Medicine and Biology”, Vol. 404, 1996, New York: Plenum Press and Springer.

As used herein, the term saponin refers to saponins obtained from natural sources, synthetic saponins, semisynthetic saponins (e.g., saponins chemically and/or enzymatically modified), and combinations thereof. In some aspects, the term saponin refers to a single saponin species, whereas in other cases (especially when referring to saponins from plant extracts), the term saponin can refer to combinations of saponins. In some aspects, a saponin used in a composition disclosed herein can be a modified saponin (e.g., chemically or enzymatically) or a saponin derivative. See, e.g., U.S. Pat. Nos. 5,443,829; 6,262,029; Wie et al. J. Agric. Food Chem., 2007, 55:8908-8913; which are herein incorporated by reference in their entireties.

Saponin containing plant: In some aspects of the present disclosure, the uric acid crystallization inhibitor comprises a saponin, e.g., an extract enriched in saponins obtained from a saponin containing plant. As used herein, the phrase “saponin containing plant” refers to plants that naturally contain saponins (e.g., Sapindus saponaria). In some aspects, the saponin-containing plant is form example, tea (Camellia sinensis), lychee (Litchi sinensis), alfalfa (Medicago sativa), chickpeas (Cicer arietinum), soybeans (Glycine max), beans (Phaseolus vulgaris), quinoa (Chenopodium quinoa), alfombrilla (Drymaria arenaroides), Christmas rose (Helleborus niger), horse chestnut trees (Aesculus hippocastanum), asparagus fern (Asparagus officinalis), licorice root (Glycyrrhiza leguminosae), soapberry (Shepherdia canadensis), soap nut (Sapindus mokorossi), Daisies (Bellis perennis), fique (Furcraea andina), agave (Agave sp.), Mojave yucca (Yucca schidigera), Quillay (Quillaja saponaria), Campions (Silene spp.), Ragged Robin (Lychnis flos-cuculi), Bracken (Pteridium aquilinum), Soap Lily (Chlorogalum pomeridianum), Ceanothus cuneatus, Yucca baccata, Yucca filamentosa, Yucca glauca, Yucca gloriosa, Yucca whipplei, Philadelphus lewisii, wild yam (Dioscorea villosa), Panax ginseng, Glycyrrhiza uralensis or combinations thereof. In some aspects, the saponin-containing plant is, for example, a Sapindaceae family plant. In some aspects, the Sapindaceae family plant is a member of the genus Sapindus. In some aspects, the member of the genus Sapindus include, for example, the species Sapindus delavayi, Sapindus detergens, Sapindus emarginatus, Sapindus laurifolius, Sapindus marginatus, Sapindus mukorossi, Sapindus oahuensis, Sapindus rarak, Sapindus saponaria, Sapindus tomentosus, Sapindus trifoliatus, and Sapindus vitiensis. In some aspects, the member of the genus Sapindus is Sapindus saponaria.

Stent: The term “stent” a metal or plastic tube inserted into the lumen of an anatomic vessel or duct to keep the passageway open. “Ureteral stents” are a specific type of “urinary tract device” used to ensure the patency of a ureter, which may be compromised, for example, by a kidney stone. This method is sometimes used as a temporary measure to prevent damage to a blocked kidney until a procedure to remove the stone can be performed. JJ stents are a particular type of ureteral stent. “Prostatic stents” another specific type of “urinary tract device” placed from the bladder through the prostatic and penile urethra to allow drainage of the bladder through the penis. This is sometimes required in benign prostatic hypertrophy.

Subject: The terms “subject,” “patient,” “individual,” and “host,” and variants thereof are used interchangeably herein and refer to any mammalian subject, including without limitation, humans, domestic animals (e.g., dogs, cats and the like), farm animals (e.g., cows, sheep, pigs, horses and the like), and laboratory animals (e.g., monkey, rats, mice, rabbits, guinea pigs and the like) for whom diagnosis, treatment, or therapy is desired, particularly humans. The methods described herein are applicable to both human therapy and veterinary applications.

Subject in need thereof: As used herein, the phrase “subject in need thereof” includes subjects, such as mammalian subjects, that would benefit from administration of one or more than one of the compositions of the present disclosure, or the application of the methods of the present disclosure, e.g., to prevent encrustation of a urinary tract device.

Urinary acidifying agent: As used herein, the term “urinary acidifying agent” refers to a compound or combination of compounds that upon ingestion by the subject lowers (acidifies) the subject's urine pH. A “lower pH” is a more acidic pH (with respect to prior measurement of urine pH).

Urinary basifying agent: As used herein, the term “urinary basifying agent” refers to a compound or combination of compounds that upon ingestion by the subject increases (basifies) the subject's urine pH. A “higher pH” is a more basic pH (with respect to prior measurement of urine pH).

Urinary tract device: As used herein, the term “urinary tract device” refers to any medical device that is inserted in the urinary tract of a subject and which is susceptible to encrustation. The term urinary tract device can denote, e.g., catheters or stents (e.g., JJ stents). In some aspects, the catheter is a urinary catheter. In some aspects, the urinary catheter is an indwelling catheter (Foley catheter). In some aspects, the urinary catheter is a balloon catheter. In some aspects, the catheter is a suprapubic catheter. In some aspects, the urinary tract device as a kidney stent (ureteric stent). In some aspects, one or both ends of the kidney stent are coiled. In some aspects, the urinary tract device is a JJ stent (JJ catheter).

### II. Methods to Reduce Urinary Device Encrustation

The present disclosure provides methods to prevent encrustation on a urinary tract device, e.g., a JJ stent, in a subject in need thereof comprising administering to the subject:
(i) at least one dose of an oral composition that decreases urine pH, e.g., if the subject's urine pH is above a pH threshold or pH range (e.g., as measured with a POC pH meter disclosed herein);
(ii) at least one dose of an oral composition that increases urine pH, e.g., if the subject's urine pH is below a pH threshold or pH range (e.g., as measured with a POC pH meter disclosed herein);
(iii) at least one dose of an oral composition that maintains urine pH, e.g., if the subject's urine pH within a pH range (e.g., as measured with a POC pH meter disclosed herein); or,
(iv) a combination thereof;
wherein the administration of the compositions to the patient reduces encrustation on the surface of the urinary tract device, e.g., a JJ stent.

In some aspects, the methods provided herein (e.g., the administration of the compositions that increase urine pH, decrease urine pH, or maintain urine pH of the present disclosure to a subject) decrease encrustation on the surface of the urinary tract device, e.g., a JJ stent, by at least 2-fold, at least 3-fold, at least 4-fold, at least 5-fold, at least 6-fold, at least 7-fold, at least 8-fold, at least 9-fold or at least 10-fold compared to the encrustation observed in control subjects. In some specific aspects, the methods provided herein (e.g., the administration of the compositions that increase urine pH, decrease urine pH, or maintain urine pH of the present disclosure to a subject) decrease encrustation on the surface of the urinary tract device, e.g., a JJ stent, by 8-fold compared to the encrustation observed in control subjects.

In some aspects, administering to a subject:
(i) at least one dose of an oral composition that decreases urine pH, e.g., if the subject's urine pH is above a pH threshold or pH range (e.g., as measured with a POC pH meter disclosed herein);
(ii) at least one dose of an oral composition that increases urine pH, e.g., if the subject's urine pH is below a pH threshold or pH range (e.g., as measured with a POC pH meter disclosed herein);
(iii) at least one dose of an oral composition that maintains urine pH, e.g., if the subject's urine pH within a pH range (e.g., as measured with a POC pH meter disclosed herein); or,
(iv) a combination thereof;
   before, after, and/or concurrently with the implantation of a urinary tract device, e.g., a JJ stent, reduces
(a) the probability of encrustation on the urinary tract device;
(b) the risk of encrustation on the urinary track device;
(c) the overall or total rate of encrustation on the urinary tract device;
(d) the rate of encrustation at the kidney end of the urinary tract device;
(e) the rate of encrustation at the bladder end of the urinary tract device;
(f) the overall or total amount of encrustation on the urinary tract device;
(g) the amount of encrustation at the kidney end of the urinary tract device;
(h) the amount of encrustation at the bladder end of the urinary tract device;
(i) encrustation on the external surface of the urinary tract device;
(j) encrustation on the internal surface of the urinary tract device;
(k) overall obstruction or occlusion of the urinary tract device;
(1) obstruction or occlusion at the kidney end of the urinary tract device;
(m) obstruction or occlusion at the bladder end of a stent of the urinary tract device; or,
(n) or a combination thereof,
   wherein the reduction in one or more of the aforementioned parameters (a) to (n) is at least about 1, at least about 1.1, at least about 1.2, at least about 1.3, at least about 1.4, at least about 1.5, at least about 1.6, at least about 1.7, at least about 1.8, at least about 1.9, at least about 2, at least about 2.1, at least about 2.2, at least about 2.3, at least about 2.4, at least about 2.5, at least about 2.6, at least about 2.7, at least about 2.8, at least about 2.9, at least about 3, at least about 3.1, at least about 3.2, at least about 3.3, at least about 3.4, at least about 3.5, at least about 3.6, at least about 3.7, at least about 3.8, at least about 3.9, at least about 4, at least about 4.1, at least about 4.2, at least about 4.3, at least about 4.4, at least about 4.5, at least about 4.6, at least about 4.7, at least about 4.8, at least about 4.9, at least about 5, at least about 5.1, at least about 5.2, at least about 5.3, at least about 5.4, at least about 5.5, at least about 5.6, at least about 5.7, at least about 5.8, at least about 5.9, at least about 6, at least about 6.1, at least about 6.2, at least about 6.3, at least about 6.4, at least about 6.5, at least about 6.6, at least about 6.7, at least about 6.8, at least about 6.9, at least about 7, at least about 7.1, at least about 7.2, at least about 7.3, at least about 7.4, at least about 7.5, at least about 7.6, at least about 7.7, at least about 7.8, at least about 7.9, at least about 8, at least about 8.1, at least about 8.2, at least about 8.3, at least about 8.4, at least about 8.5, at least about 8.6, at least about 8.7, at least about 8.8, at least about 8.9, at least about 9, at least about 9.1, at least about 9.2, at least about 9.3, at least about 9.4, at least about 9.5, at least about 9.6, at least about 9.7, at least about 9.8, at least about 9.9, or at least about 10-fold compared to the level/rate/magnitude of the one or more of the aforementioned parameters (a) to (n) observed/measured in control subjects. In some aspects, the reduction is between 1-fold and 10-fold, or between 2-fold and 10-fold, or between 3-fold and 10-fold, or between 4-fold and 10-fold, or between 5-fold and 10-fold, or between 6-fold and 10-fold, or between 7-fold and 10-fold, or between 8-fold and 10-fold, or between 9-fold and 10-fold, or between 2-fold and 4-fold, or between 4-fold and 6-fold, or between 6-fold and 8-fold, or between 8-fold and 10-fold, or between 1-fold and 3-fold, or between 3-fold and 5-fold, or between 5-fold and 7-fold, or between 7-fold and 9-fold compared to the level/rate/magnitude of the one or more of the aforementioned parameter (a) to (n) observed/measured in control subjects.

As used herein the term “control subject” refers to a subject who has had an urinary tract device implanted and kept in place for a period of time, but has not had any intervention to regulate urine pH as disclosed herein. Thus, the term “control subject” refers to a subject who has not been administered the compositions that increase urine pH, decrease urine pH, or maintain urine pH disclosed herein.

In some aspects, the “control subject” is the same subject who is administered the compositions of the present disclosure according to the methods of the present disclosure. For example, encrustation can be measured on urinary tract devices (e.g., stents) implanted and subsequently extracted from a subject, and the effect of the administration of the compositions of the present disclosure according to the methods of the present disclosure on encrustation on a newly implanted urinary tract device can be quantified with respect to the levels of encrustation observer on prior devices. Analogous comparisons can be made to evaluate the effect of the compositions and methods disclosed herein on other parameters, e.g., pain, discomfort, or tissue damage (i.e., comparing pain, discomfort, or tissue damage in prior device extractions, with pain, discomfort, or tissue damage experience after applying the compositions and methods disclosed herein). In some aspects of the methods disclosed herein, the term “control subject” can be used interchangeably with the term “control conditions.” The term “control conditions” refers to conditions under which a urinary tract device is implanted and/or extract from a subject without administration of the compositions disclosed herein and/or application of the methods disclosed herein.

A “control subject” can also be, e.g., a population of individuals selected a reference population in a clinical trial. Accordingly, the magnitude of the effects resulting from the application of the methods disclosed herein can also be relative to the average effect observed in a reference population of untreated subjects.

In some aspects, the methods provided herein (e.g., the administration of the compositions that increase urine pH, decrease urine pH, or maintain urine pH to the subject) decrease encrustation levels to at least about 90%, at least about 85%, at least about 80%, at least about 75%, at least about 70%, at least about 65%, at least about 60%, at least about 55%, at least about 50%, at least about 45%, at least about 40%, at least about 35%, at least about 30%, at least about 25%, at least about 20%, at least about 15%, at least about 10%, at least about 5%, or about 0% of the amount of encrustation observed in control subjects or under control conditions.

In some aspects, the methods provided herein (e.g., the administration of the compositions that increase urine pH, decrease urine pH, or maintain urine pH to the subject) decreases encrustation by at least about 10%, at least about 15%, at least about 20%, at least about 25%, at least about 30%, at least about 35%, at least about 40%, at least about 45%, at least about 50%, at least about 55%, at least about 55%, at least about 60%, at least about 70%, at least about 75%, at least about 80%, at least about 85%, at least about 90%, at least about 95%, or 100% compared the encrustation observed in control subjects or under control conditions.

In some aspects, the oral composition that decreases urine pH comprises a urinary acidifying agent. In other aspects, the oral composition that decreases urine pH comprises a crystallization inhibitor. In some aspects, the oral composition that decreases urine pH comprises a urinary acidifying agent and a crystallization inhibitor, e.g., calcium phosphate.

In some aspects, the urinary acidifying agent comprises an amino acid or a combination thereof (e.g., methionine or arginine). In some aspects, the urinary acidifying agent comprises L-methionine.

In some aspects, the urinary acidifying agent is selected from a portion or an extract of the plant genus Vaccinium, pharmaceutically acceptable ammonium salts, arginine or any of its pharmaceutically acceptable salts, cysteine, or any of its pharmaceutically acceptable salts, phenazopyridine or any of its pharmaceutically acceptable salts, or combinations thereof.

Vaccinium is a common and widespread genus of shrubs in the heath family (Ericaceae). The genus contains about 450 species. The fruit of many species are eaten by human, e.g, cranberry, blueberry, bilberry (whortleberry), lingonberry (cowberry) and huckleberry. Like many other ericaceous plants, they are generally restricted to acidic soils. In some aspects, the Vaccinium plant is V. macrocarpon (American cranberry), V. oxycoccos (Common cranberry), V. microcarpum (Small bog cranberry) V. erythrocarpum (Southern mountain cranberry), V. arboretum (Sparkleberry), V. crassifolium (Creeping blueberry), V. moupinense (Himalayan blueberry), V. angustifolium (Lowbush blueberry), V. boreale (Northern blueberry), V. caesariense (New Jersey blueberry), V. caespitosum (Dwarf blueberry), V. corymbosum (Highbush blueberry), V. darrowii (Evergreen blueberry), V. elliottii (Elliott's blueberry), V. fuscatum (Black highbush blueberry), V. myrsinites (Evergreen blueberry), V. myrtilloides (Canadian blueberry), V. pallidum (Dryland blueberry), V. virgatum (Rabbiteye blueberry), V. koreanum (Korean blueberry), V. myrtillus (Common bilberry).

In some aspects, the urinary acidifying agent is selected from cranberry, ammonium chloride, L-arginine, L-cysteine, or combinations thereof. In some aspects, the urinary acidifying agent is L-methionine.

In some aspects, the calcium phosphate crystallization inhibitor comprises phytic acid, a salt thereof, or a combination thereof. In some aspects, the oral composition that decreases urine pH comprises phytate in any form (e.g., pure, as a plant extract, as a plant part, or a combination thereof) disclosed in the present disclosure.

In some aspects, the oral composition that decreases urine pH comprises L-methionine, rice husk extract (rice bran extract) enriched in phytic acid magnesium and calcium salts, zinc gluconate, and vitamin A. In some aspects, the oral composition that decreases urine pH is a composition disclosed in U.S. Appl. Publ. No. US2017/0106032, which is herein incorporated by reference in its entirety.

In one specific aspect, the oral composition that decreases urine pH comprises American cranberry (200 mg), L-arginine (180 mg), L-cysteine (180 mg), and calcium magnesium phytate (120 mg), per dose. In another specific aspect, the oral composition that decreases urine pH comprises American cranberry (250 mg), L-arginine (150 mg), L-cysteine (150 mg), and calcium magnesium phytate (150 mg), per dose. In another aspect, the oral composition that decreases urine pH comprises American cranberry (220 mg), L-arginine (160 mg), L-cysteine (160 mg), and calcium magnesium phytate (180 mg), per dose. In another aspect, the oral composition that decreases urine pH comprises American cranberry (230 mg), L-arginine (130 mg), L-cysteine (130 mg) and potassium phytate (130 mg), per dose. The composition that decreases urine pH can be administered as one dose or multiple doses until the desired effect is reached.

It is know that at pH values higher than 6.2, urine is supersaturated with respect to calcium phosphate, which can crystallize as brushite or hydroxyapatite. Accordingly, in some aspects, the subject is administered at least one dose of an oral composition that decreases urine pH disclosed herein, if the subject's urine pH is above a pH 6.2 threshold, or above pH 6.2 in a range that has such pH as its upper limit.

In some aspects, the oral composition that increases urine pH comprises a urinary basifying agent. In some aspects, the oral composition that increases urine pH comprises a uric acid crystallization inhibitor. In some aspects, the oral composition that increases urine pH comprises a uric acid basifying agent and a uric acid crystallization inhibitor. In some aspects, the urinary basifying agent comprises citric acid, salts thereof, or combinations thereof. In some aspects, the urinary basifying agent comprises potassium citrate, magnesium citrate, sodium bicarbonate, potassium bicarbonate, or a combination thereof.

In some aspects, the urinary basifying agent comprises at least one pohyhydroxycarboxylic acid. In some aspects, the polyhydroxycarboxylic acid is selected from ethyeleneaminetetraacetic acid (EDTA), isocitric acid, malic acid, succinic acid, or a combination thereof.

In some aspects, the uric acid crystallization inhibitor comprises at least one saponin and/or at least one glycosaminoglycan, or a combination thereof. In some aspects, saponins and glycosaminoglycans are derived from part of a plant or animal species rich in these compounds, or a plant or animal extract taken from said species rich in these compounds.

In some aspects, the oral composition that increases urine pH comprises potassium citrate, magnesium citrate, a saponin, zinc gluconate, and vitamin A. In some aspects, the oral composition that increases urine pH is a composition disclosed in International App. Publ. No. WO2015150609, which is herein incorporated by reference in its entirety.

In some specific aspects, the oral composition that increases urine pH comprises potassium citrate (300 mg), magnesium citrate (250 mg), ginseng extract with >30% in saponins (150 mg), per dose. In another specific aspect, the oral composition that increases urine pH comprises potassium citrate (350 mg), magnesium citrate (200 mg), and glycyrrhizic acid (160 mg), per dose. In another specific aspect, the oral composition that increases urine pH comprises sodium bicarbonate (4.20 mg), potassium bicarbonate (5.10 mg) and Indian chestnut seed extract equivalent to 50 mg of aescin per dose. In yet another specific aspect, the oral composition that increases urine pH comprises sodium bicarbonate (6 mg), potassium bicarbonate (7 mg), and aescin (60 mg), per dose. The composition that increases urine pH can be administered as one dose or multiple doses until the desired effect is reached.

Urinary pH values of below 5.5 can lead to the formation of uric acid and calcium oxalate deposits. Accordingly, in some aspects, the subject is administered at least one dose of an oral composition that increases urine pH, if the subject's urine pH is below a pH 5.5 threshold, or below pH 5.5 in a range that has such pH as its lower limit.

In some aspects, the oral composition that maintains urine pH comprises (i) a calcium phosphate crystallization inhibitor, and (ii) a magnesium salt, magnesium hydroxide, magnesium oxide, or a combination thereof. In some aspects, the calcium phosphate crystallization inhibitor comprises phytic acid, a salt thereof, or a combination thereof. In some aspects, the oral composition that maintains urine pH comprises magnesium oxide. In some aspects, the magnesium in the composition that maintains urine pH can be supplied as, for example, magnesium hydroxide, magnesium oxide, magnesium citrate, magnesium stearate, magnesium carbonate, magnesium chloride, magnesium sulfate, or a combination thereof.

In some aspects, the oral composition that maintains urine pH comprises between 40 and 50% by weight of phytic acid or its salts. In some aspects, the oral composition that maintains urine pH comprises between 25 and 40% by weight of magnesium in the form of salt, hydroxide or oxide.

In some aspects, the oral composition that maintains urine pH further comprises at least one polyphenol. In some aspects, the oral composition that maintains urine pH comprises between 10-30% by weight of polyphenols. In some aspects, the oral composition that maintains urine pH is a composition disclosed in U.S. Appl. Publ. No. US2017/0128469, which is herein incorporated by reference in its entirety.

In some aspects, the composition that maintains urine pH comprises rice husk extract (rice bran extract) enriched in phytic acid magnesium and calcium salts, magnesium oxide, grape seed extract enriched in polyphenols, zinc gluconate, and vitamin A. In one aspect, the composition that maintains urine pH comprises calcium-magnesium phytate (300 mg), magnesium citrate (250 mg), and epicatechin (100 mg), per dose. In another aspect, the composition that maintains urine pH comprises calcium-magnesium phytate (300 mg), magnesium citrate (250 mg) and catechin (150 mg), per dose. In yet another aspect, the composition that maintains urine pH comprises brown rice extract equivalent to an amount of phytate of 250 mg, magnesium oxide (150 mg), and black grape seed extract equivalent to an amount of polyphenols of 150 mg, per dose. In another aspect, the composition that maintains urine pH comprises locust bean germ extract and dry wheat extract equivalent to an amount of phytate of 150 mg, magnesium oxide (100 mg), and white grape seed extract equivalent to an amount of polyphenols of 90 mg, per dose. In another aspect, the composition that maintains urine pH comprises calcium-magnesium phytate (200 mg), magnesium citrate (200 mg) and quercetin (100 mg), per dose. The composition that maintains urine pH can be administered as a single dose or multiple doses until the desired effect is reached.

In some aspects, the subject is administered (e.g., self-administered) at least one dose of an oral composition that maintains urine pH, if the subject's urine pH is in a range between pH 5.5 and pH 6.2.

In some aspects, the compositions of the present disclosure, i.e., oral compositions that increase urine pH, decrease urine pH, or maintain urine pH, are nutraceuticals or functional foods, i.e., foods that have a beneficial effect on health. Nutraceuticals can be used in nutritional mixtures in the food industry and in the pharmaceutical industry. Thus, compositions of the present disclosure can be administered for example as pills, gelcaps, powders, syrups, lozenges, and other formats for oral administration known in the art, or they can be administered, for example, as nutraceuticals, e.g., as components of foodstuffs or beverages.

In some aspects, the phytic acid, a salt thereof, or combination thereof in the compositions of the present disclosure is administered as a plant extract. In some aspects, the plant extract is a rice extract. In some aspects, the rice extract is a rice husk extract (rice bran extract). In some specific aspects, the rice husk extract is enriched in phytin. In some aspects, the phytin is a magnesium and/or calcium salt of phytic acid.

In some aspects the phytic acid, a salt thereof, or combination thereof in the compositions of the present disclosure can be used, e.g., (i) in free form as a pure substance, (ii) as an extract at least one plant species containing them, such as, for example, extracts of white or brown rice, (iii) as a plant part containing them, such as the germs or the external parts of the wheat, oat, soy, almond, locust bean, etc. grains or fruits, or (iv) a combination thereof.

In some aspects, the phytic acid, salt thereof, or combination thereof in the compositions of the present disclosure is administered as a purified preparation.

In some aspects, the purified phytic preparation comprises at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, or at least about 99% phytic acid.

In some aspects, the purified phytate preparation comprises at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, or at least about 99% phytate.

In some aspects, the purified phytin preparation comprises at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, or at least about 99% phytin.

In some aspects, the at least one saponin in a composition of the present disclosure is administered as a plant extract. In some aspects, the at least one saponin is administered as a purified preparation. In some aspects, the purified saponin preparation comprises at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, or at least about 99% pure saponin (or saponins).

In some aspects, the at least one polyphenol in a composition of the present disclosure is administered as a plant extract. In some aspects, the plant extract is a grape extract. In some aspects, the grape extract is a grape seed extract. In some aspects, the at least one polyphenol is administered as a purified preparation. In some aspects, the purified polyphenol preparation comprises at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, or at least about 99% pure polyphenol (or polyphenols).

The present disclose provides methods that can be applied to any medical device inserted in the urinary tract that is susceptible to encrustation. In some aspects, the urinary tract device is a bladder stent or kidney stent. In some particular aspects, the kidney stent is a JJ stent. In some aspects, the methods disclosed herein can prevent or reduce encrustation on the surface of the medical device, e.g., the external surface of a catheter or stent (e.g., a JJ stent). In some aspects, the methods disclosed herein can prevent or reduce encrustation on an internal surface of the medical device, e.g., the internal surface of a catheter or stent (e.g., a JJ stent). It is known in the art that encrustation on the internal (luminal) surface of a catheter or stent (e.g., a JJ stent) can result in obstruction or occlusion.

In some aspects, the methods disclosed herein further comprise the measurement of urine pH by the subject, e.g., using a self-diagnostic pH measuring device (e.g., h a POC pH meter disclosed herein). In some aspects, the self-diagnostic pH measuring device comprises a sensor disclosed in U.S. Pat. No. 4,874,499, which is herein incorporated by reference in its entirety. In other aspects, the self-diagnostic pH measuring device comprises a sensor disclosed in U.S. Pat. No. 10,067,085, which is herein incorporated by reference in its entirety. In some aspects, the self-diagnostic pH measuring device is a device disclosed in U.S. App. Publ. No. US 2016/0061813, which is herein incorporated by reference in its entirety. In some aspects, the subject can measure his or her urine pH and adjust the administration of the compositions of the present disclosure, e.g., (i) the type of composition of the present disclosure, (ii) the dosage (e.g., number of doses, such as pills, ingested at a certain time), (iii) the frequency of dosage, or (iv) a combination thereof, according to a clinician's advice (e.g., a physician) and/or instructions provided as part of a kit, product of manufacture, prescription, or a companion web site or application.

The present disclosure also provides methods to facilitate the removal of a urinary tract device (e.g., a JJ stent) in a subject in need thereof comprising administering to the subject: (i) at least one dose of an oral composition that decreases urine pH, e.g., if the subject's urine pH is above a pH threshold or pH range (e.g., as measured with a POC pH meter disclosed herein); (ii) at least one dose of an oral composition that increases urine pH, e.g., if the subject's urine pH is below a pH threshold or pH range (e.g., as measured with a POC pH meter disclosed herein); (iii) at least one dose of an oral composition that maintains urine pH, e.g., if the subject's urine pH within a pH range (e.g., as measured with a POC pH meter disclosed herein); or, (iv) a combination thereof; wherein the administration of the compositions to the subject prevents or reduces encrustation over time on the surface of the urinary tract device, and the reduced encrustation facilitates the removal of the urinary tract device.

The present disclosure also provides methods to reduce encrustation on the surface (e.g., the external surface and/or the internal surface) of a urinary tract medical device, e.g., a JJ stent, in a subject with respect to the level of encrustation observed on the surface of a corresponding urinary tract medical device in control subjects comprising administering to the subject: (i) at least one dose of an oral composition that decreases urine pH, e.g., if the subject's urine pH is above a pH threshold or pH range (e.g., as measured with a POC pH meter disclosed herein); (ii) at least one dose of an oral composition that increases urine pH, e.g., if the subject's urine pH is below a pH threshold or pH range (e.g., as measured with a POC pH meter disclosed herein); (iii) at least one dose of an oral composition that maintains urine pH, e.g., if the subject's urine pH within a pH range (e.g., as measured with a POC pH meter disclosed herein); or, (iv) a combination thereof.

The present disclosure also provides methods to reduce the amount of encrustation on the surface (e.g., the external surface and/or the internal surface) of a urinary tract medical device, e.g., a JJ stent, in a subject with respect to the level of encrustation observed on the surface of a corresponding urinary tract medical device in control subjects comprising administering to the subject: (i) at least one dose of an oral composition that decreases urine pH, e.g., if the subject's urine pH is above a pH threshold or pH range (e.g., as measured with a POC pH meter disclosed herein); (ii) at least one dose of an oral composition that increases urine pH, e.g., if the subject's urine pH is below a pH threshold or pH range (e.g., as measured with a POC pH meter disclosed herein); (iii) at least one dose of an oral composition that maintains urine pH, e.g., if the subject's urine pH within a pH range (e.g., as measured with a POC pH meter disclosed herein); or, (iv) a combination thereof.

The present disclosure also provides methods to reduce the rate of encrustation on the surface (e.g., the external surface and/or the internal surface) of a urinary tract medical device, e.g., a JJ stent, in a subject with respect to the level of encrustation observed on the surface of a corresponding urinary tract medical device in control subjects comprising administering to the subject: (i) at least one dose of an oral composition that decreases urine pH, e.g., if the subject's urine pH is above a pH threshold or pH range (e.g., as measured with a POC pH meter disclosed herein); (ii) at least one dose of an oral composition that increases urine pH, e.g., if the subject's urine pH is below a pH threshold or pH range (e.g., as measured with a POC pH meter disclosed herein); (iii) at least one dose of an oral composition that maintains urine pH, e.g., if the subject's urine pH within a pH range (e.g., as measured with a POC pH meter disclosed herein); or, (iv) a combination thereof.

The present disclosure also provides methods to reduce tissue damage due to urinary tract medical device encrustation during the extraction of the urinary tract medical device, e.g., a JJ stent, from a subject with respect to the tissue damage observed in control subjects comprising administering to the subject: (i) at least one dose of an oral composition that decreases urine pH, e.g., if the subject's urine pH is above a pH threshold or pH range (e.g., as measured with a POC pH meter disclosed herein); (ii) at least one dose of an oral composition that increases urine pH, e.g., if the subject's urine pH is below a pH threshold or pH range (e.g., as measured with a POC pH meter disclosed herein); (iii) at least one dose of an oral composition that maintains urine pH, e.g., if the subject's urine pH within a pH range (e.g., as measured with a POC pH meter disclosed herein); or, (iv) a combination thereof.

The present disclosure also provides methods to reduce pain and/or discomfort (e.g., duration and/or intensity thereof) due to urinary tract medical device encrustation during the extraction of the urinary tract medical device, e.g., a JJ stent, from a subject with respect to the pain and/or discomfort (e.g., duration and/or intensity thereof) observed in control subjects comprising administering to the subject: (i) at least one dose of an oral composition that decreases urine pH, e.g., if the subject's urine pH is above a pH threshold or pH range (e.g., as measured with a POC pH meter disclosed herein); (ii) at least one dose of an oral composition that increases urine pH, e.g., if the subject's urine pH is below a pH threshold or pH range (e.g., as measured with a POC pH meter disclosed herein); (iii) at least one dose of an oral composition that maintains urine pH, e.g., if the subject's urine pH within a pH range (e.g., as measured with a POC pH meter disclosed herein); or, (iv) a combination thereof.

The present disclosure also provides methods to improve the quality of life of a subject with a urinary tract medical device, e.g., a JJ stent, by reducing, e.g., pain or tissue damage due to urinary tract medical device encrustation comprising administering to the subject: (i) at least one dose of an oral composition that decreases urine pH, e.g., if the subject's urine pH is above a pH threshold or pH range (e.g., as measured with a POC pH meter disclosed herein); (ii) at least one dose of an oral composition that increases urine pH, e.g., if the subject's urine pH is below a pH threshold or pH range (e.g., as measured with a POC pH meter disclosed herein); (iii) at least one dose of an oral composition that maintains urine pH, e.g., if the subject's urine pH within a pH range (e.g., as measured with a POC pH meter disclosed herein); or, (iv) a combination thereof.

Quality of life can be estimated, e.g., using the self-perceived quality-of-life scale, which is a psychological assessment instrument which is based on a comprehensive theory of the self-perceived quality of life (SPQL) and provides a multi-faceted measurement of health-related and non-health-related aspects of well-being. Quality of life can also be assessed using alternative scales known in the art.

The present disclosure also provides methods to prevent or reduce the risk of stent, e.g., a JJ stent, blockage or occlusion (e.g., at either end or both ends of a stent) in a subject due to encrustation with respect to the rate of blockage or occlusion observed in control subjects comprising administering to the subject: (i) at least one dose of an oral composition that decreases urine pH, e.g., if the subject's urine pH is above a pH threshold or pH range (e.g., as measured with a POC pH meter disclosed herein); (ii) at least one dose of an oral composition that increases urine pH, e.g., if the subject's urine pH is below a pH threshold or pH range (e.g., as measured with a POC pH meter disclosed herein); (iii) at least one dose of an oral composition that maintains urine pH, e.g., if the subject's urine pH within a pH range (e.g., as measured with a POC pH meter disclosed herein); or, (iv) a combination thereof.

In some aspects, the composition and methods disclosed herein can be applied as a co-therapy, e.g., in the treatment of diseases or conditions that require the implantation of a urinary tract medical device in the urinary tract of a patient in need thereof.

In some aspects, the compositions and methods disclosed herein can be applied before (e.g., pre-surgical), during, or after (e.g., post-surgical), implantation of a urinary tract medical device in the urinary tract of a patient in need thereof.

In some aspects, the compositions and methods disclosed herein can be applied in combination with antibiotic therapy, e.g., co-administration with antibiotics known in the art generally used to treat urinary tract infections. In some aspects, the antibiotic comprises penicillin (e.g., ampicillin, amoxicillin, mecillinam, or amoxicillin clavulanate), sulfonamide (e.g., trimethoprim-suldamethoxazone), cephalosporin (e.g., cefadroxil, cefazolin, cephalexin, cephradine, cefaclor, cefamandole, cefotetan, cefoxitin, cefuroxime, cefdinir, cefixime, cefoperazone, cefazidime, ceftriaxone, or cefepime), fluoroquinolone (e.g., ciprofloxacin, enoxacin, gatigloxacin, levofloxacin, norfloxacin, or ofloxacin), aminoglycoside (e.g., gentamicin), nitrofurantoin, forfomycin, or any combination thereof. In some aspects, the antibiotic is administered before, after, or concurrently with the compositions and methods disclosed herein. In some aspects, antibiotics are applied before, during, or after, the implantation of a urinary tract medical device, e.g., a JJ stent, in the urinary tract of a patient in need thereof.

In some aspects, the compositions and methods disclosed herein prevent or reduce encrustation (e.g., total amount encrustation or rate of encrustation) at the kidney end of a stent (e.g., a JJ stent), at the bladder end of a stent (e.g., a JJ stent), or both at the kidney end and bladder end of a tent (e.g., a JJ stent).

In some aspects, the methods disclosed herein prevent or reduce encrustation (e.g., total amount encrustation or rate of encrustation) over the surface of the entire stent (e.g., a JJ stent).

In some aspects, the methods disclosed herein prevent or reduce encrustation (e.g., total amount encrustation or rate of encrustation) preferentially at the kidney end of the stent (e.g., a JJ stent). In other aspects, the methods disclosed herein prevent or reduce encrustation (e.g., total amount encrustation or rate of encrustation) preferentially at the bladder end of a stent (e.g., a JJ stent).

In some aspects, all the doses of oral compositions that increase, decrease, or maintain urine pH disclosed herein have the same composition (i.e., the same formulation) during the duration of the treatment. For example, a particular oral composition of the present disclosure (e.g., a composition that decreases urine pH) can be used throughout the entire treatment. In other cases, a first certain oral composition of the present disclosure (e.g., a composition that decreases urine pH) can be replaced by a second corresponding composition of the present disclosure (i.e., another composition that decreases urine pH) during the treatment. In some aspects, the first and second composition have the same urinary acidifying agent or combination thereof. In some aspects, the first and second composition have the same urinary pH modifying agent (e.g., an acidifying agent) agent or combination thereof but at different concentrations and/or ratios. In some aspects, the first and second composition differ in at least one urinary pH modifying agent (e.g., an acidifying agent). In some aspects, the first and second composition comprise different urinary pH modifying agents (e.g., an acidifying agent).

In other aspects, the doses of oral compositions that increase, decreases, or maintain urine pH can vary during the duration of the treatment. In some aspects, the subject is administered at least one loading dose larger than the rest of doses at the beginning of the treatment. In some aspects, the subject is administered at least one maintenance dose during the treatment after the administration of a loading dose.

In some aspects, the compositions of the present disclosure are administered as a pre-treatment before the insertion of the urinary tract device (e.g., a JJ stent). In some aspects, the compositions of the present disclosure are administered according to the methods disclosed herein until the extraction of the urinary tract device (e.g., a JJ stent).

In some aspects, the compositions of the present disclosure are administered at least once a day, at least twice a day, at least three times a day, or at least four times a day. In some aspects, the compositions of the present disclosure are administered for at least about 1 week, at least about 2 weeks, at least about 3 weeks, at least about 4 weeks, at least about 5 weeks, at least about 6 weeks, at least about 7 weeks, at least about 8 weeks, at least about 9 weeks, at least about 10 weeks, at least about 11 weeks, at least about 12 weeks, at least about 13 week, at least about 14 weeks, at least about 15 weeks, or at least about 16 weeks.

In some aspect, urine pH is measured 1 time per day, 2 times per day, 3 times per day, 4 times per day, 5 times per day, or 6 times per day. In some aspects, urine pH is measured immediately before administration of a composition of the present disclosure. In some aspects, urine pH is measured after the administration of a composition of the present disclosure, e.g., after at least about 30 minutes, at least about 1 hour, at least about 2 hours, at least about 3 hours, at least about 4 hours, at least about 5 hours, at least about 6 hours, at least about 7 hours, at least about 8 hours, at least about 9 hours, at least about 10 hours, at least about 11 hours, at least about 12 hours, at least about 13 hours, at least about 14 hours, at least about 15 hours, at least 16 hours, at least about 17 hours, at least about 18 hours, at least about 19 hours, at least about 20 hours, at least about 21 hours, at least about 22 hours, at least about 23 hours, or at least about 24 hours.

In some aspects, the methods of the present disclosure can effectively reduce struvite encrustation on the surface of the urinary tract devices, e.g., JJ stents. Struvite (magnesium ammonium phosphate) is a phosphate mineral. Struvite urinary stones and struvite encrustation readily form in animal and humans that are infected with ammonia-producing organisms. Struvite encrustation is generally caused and potentiated by bacterial infection that hydrolyzes urea to ammonium and raises urine pH to neutral or alkaline values. Urea-splitting organisms include Proteus, Pseudomonas, Klebsiella, Staphylococcus, and Mycoplasma. Struvite kidney stones are common in humans, cats, and dogs (in fact, struvite is the most common mineral found in urinary tract stones in dogs), and also in enteroliths (intestinal concretions) in horses.

Unexpectedly, the methods of the present disclosure have been found to be particularly effective in preventing or reducing struvite encrustation on urinary tract devices (e.g., JJ stents). In particular, the compositions and methods of the present disclosure have been found to be particularly effective to prevent and/or reduce struvite encrustation developed in the absence of infection by urea-splitting organisms. Accordingly, the compositions of the present disclosure and any of the methods disclosed herein can be applied wherein the encrustation comprises, consists, or consists essentially of struvite deposits. In one aspect, the compositions of the present disclosure and any of the methods disclosed herein can be applied wherein the encrustation comprises, consists, or consists essentially of struvite in the absence of a urinary tract infection, e.g., a bacterial infection that hydrolyzes urea to ammonium.

In some aspects, the present disclosure provides methods to prevent or reduce the formation of struvite deposits (e.g., kidney stones in cats or dogs, or enteroliths in horses), or ameliorate symptoms associates with such deposits (e.g., tissue damage or pain) comprising administering to the subject (e.g., a veterinarian subject such as a cat, a dog or a horse) (i) at least one dose of an oral composition that decreases urine pH, e.g., if the subject's urine pH is above a pH threshold or pH range; (ii) at least one dose of an oral composition that increases urine pH, e.g., if the subject's urine pH is below a pH threshold or pH range; (iii) at least one dose of an oral composition that maintains urine pH, e.g., if the subject's urine pH within a pH range; or, (iv) a combination thereof. In some aspects, pH is measured using a portable pH meter disclosed herein.

### III. Kits

The present disclosure also provides kits or products of manufacture comprising (i) an oral composition that decreases urine pH (e.g., LIT-CONTROL DEVICARE® pH Down), (ii) an oral composition that increases urine pH (e.g., LIT-CONTROL DEVICARE® pH Up), (iii) an oral composition that maintains urine pH (e.g., LIT-CONTROL DEVICARE® pH Balance), or (iv) a combination thereof, and instructions to administer (e.g., self-administer) the compositions according to the methods disclosed herein.

The present disclosure also provides a kit comprising a point-of-care pH measuring device (e.g., LIT-CONTROL DEVICARE® pH Meter) and instructions to administer (i) an oral composition that decreases urine pH (e.g., LIT-CONTROL DEVICARE® pH Down), (ii) an oral composition that increases urine pH (e.g., LIT-CONTROL DEVICARE® pH Up), (iii) an oral composition that maintains urine pH (e.g., LIT-CONTROL DEVICARE® pH Balance), or (iv) a combination thereof according to the methods disclosed herein.

The present disclosure also provides kits or products of manufacture comprising (i) an oral composition that decreases urine pH (e.g., LIT-CONTROL DEVICARE® pH Down), (ii) an oral composition that increases urine pH (e.g., LIT-CONTROL DEVICARE® pH Up), (iii) an oral composition that maintains urine pH (e.g., LIT-CONTROL DEVICARE® pH Balance), or (iv) a combination thereof, plus a point-of-care pH measuring device (e.g., LIT-CONTROL DEVICARE® pH Meter), and instructions to administer the compositions according to the methods disclosed herein.

In some aspects, the instructions are, e.g., provided as a brochure or insert, printed on the packaging, printed on a label, or provided electronically, for example, via a web site.

It is to be appreciated that the Detailed Description section, and not the Summary and Abstract sections, is intended to be used to interpret the claims. The Summary and Abstract sections may set forth one or more but not all exemplary embodiments of the present invention as contemplated by the inventor(s), and thus, are not intended to limit the present invention and the appended claims in any way.

The breadth and scope of the present invention should not be limited by any of the above-described exemplary embodiments, but should be defined only in accordance with the following claims and their equivalents.

All of the references cited above, as well as all references cited herein, are incorporated herein by reference in their entireties.

The following examples are offered by way of illustration and not by way of limitation.

### EXAMPLES

### Example 1

### Mechanisms of Encrustation of Urinary Tract Stents

Introduction

The development of encrustations on urinary stents is an important problem related to the use of these renal stents. It must be taken into account that as a consequence of the development of the scale, in addition to its obstruction, important problems can be generated when they are removed. The most frequent obstructions that have been described more frequently, are due to the blockage taking place due to crystalline deposits formed as a consequence of the increase in urinary pH by the action of urease-producing bacteria. The most commonly found bacteria being Proteus mirabilis.

However, it is important to consider that not all the crystalline deposits that are generated on the stents are a consequence of bacterial infections. Although less frequent, have also been observed important deposits of hydroxyapatite/calcium oxalate dihydrate, brushite and also uric acid.

The main objective of this example is to study in detail the concretions which are developed on the surface of a large series of stents obtained in a study to avoid stent infection, and establish the mechanisms and stages through which their development takes place. The better knowledge of their formation mechanism will allow a better approach to avoid their development. On the other hand it should be taken into account that these encrustations develop inside the renal system, in close contact with urine. Therefore, important aspects related to the mechanism of renal stone formation can also be derived from such studies.

Materials and Methods

This study includes 96 stents that came from a Multicenter, Controlled and Randomized Clinical Trial to Evaluate the Combined use of a Medical Device and a Food Supplement in the Control of Urinary pH in Patients With an Implanted Double Pigtail Stent (Clinical trial reference NCT03343275).

Patients were evaluated for urinary pH (by pH-meter) before, during and at the end of the study. A urinary culture was performed prior to the stent implantation to verify the absence of urinary infection. Stents were implanted for about two months that the trial lasted.

Encrustation Characterization:

The encrustations were evaluated in the renal part and in the bladder part, and according to their magnitude they were classified by stereoscopic microscopy (Optomic) in: 0 (No encrustation); 1 (Sporadic encrustation, thickness less than 1 mm); 2 (Wide area of calcifications, thickness between 1 to 2 mm); 3 (Total coating, thickness less than 2 mm); 4 (Total obstruction, thickness more than 2 mm).

The type of deposit generated on the stents, the presence of bacteria and the size and nature of the crystals formed was identified by scanning electron microscopy (Hitachi S 3400 N), coupled to a microanalysis by X-ray dispersive energy (Bruker AXS GmbH, Karlsruhe, Germany).

Determination of the Amount of Calcium Deposited on the Stent:

Five stents classified as 1 and four stents classified as 4 were selected at random. The selected stents were fragmented in one of its extremes (its length was measured) and introduced into a tube with hydrochloric acid. This solution, once filtered, was the one used to determine calcium colorimetrically with the Arsenazo III method (Michaylova V, et al., Anal Chim Acta 1971; 53: 194-198) The results obtained are shown in Table 1.

TABLE 1
Amount of calcium deposited on the stent.

| Magnitude of the scale | Stent extreme Id | Amount of calcium |
|---|---|---|
| Type 1 | 49 bladder | 0.95 nmol/cm |
| | 59 bladder | 1.21 nmol/cm |
| | 43 renal | 0.84 nmol/cm |
| | 49 renal | 0.42 nmol/cm |
| | 50 renal | 0.85 nmol/cm |
| Type 4 | 41 bladder | 330 nmol/cm |
| | 3 bladder | 346 nmol/cm |
| | 72 bladder | 244 nmol/cm |
| | 3 renal | 329 nmol/cm |

Results

The total amount of calcium deposited in stents with encrustation scores of 4 was one thousand times greater than the amount in stents with score 1 (Table 1).

Tables 2 and 3 summarize the types of scale and the magnitude of the deposits. As can be observed, in the 28.3% of the stents no deposit was observed in the bladder part, while in the renal part there were no deposits in 40.4%. In 53.5% of the bladder part and in 46.5% of the renal part, slight deposits of thickness 1-10 microns were observed.

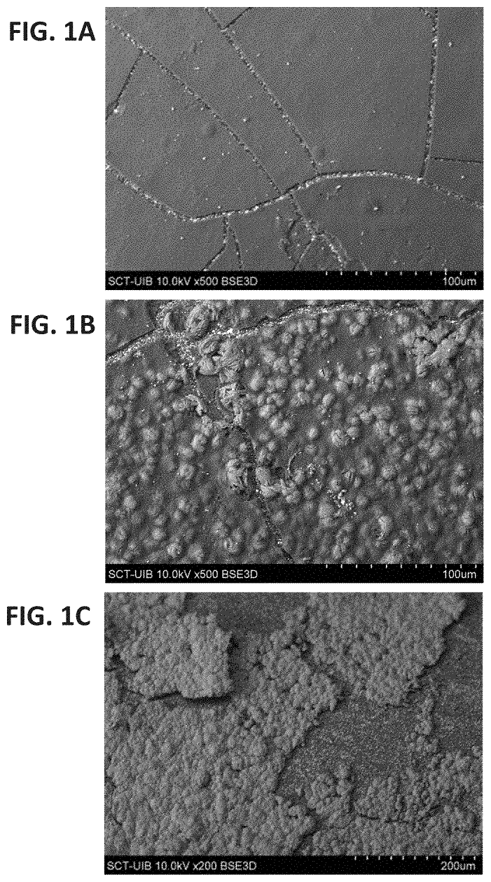

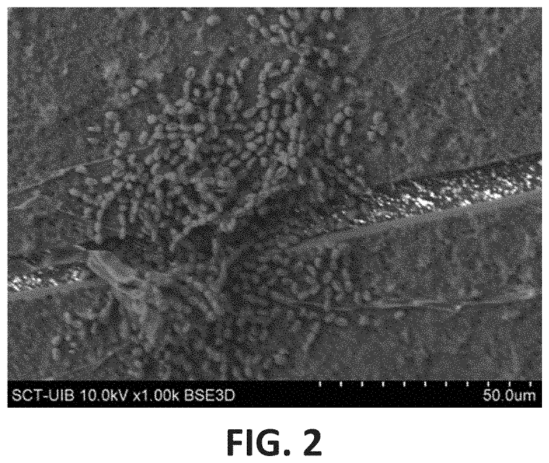

These deposits consist mainly of only organic matter (12.1% bladder part—8.1% renal part) or small crystals of calcium oxalate monohydrate (COM) developed on a layer of organic matter, as can be seen in FIG. 1A-1C. In addition, bacteria were on the surface of the bladder part in 4.0% of the stents and on the renal part in 2.0% of the stents. In all cases, bacteria were on top of the layer of the initially deposited organic matter (FIG. 2).

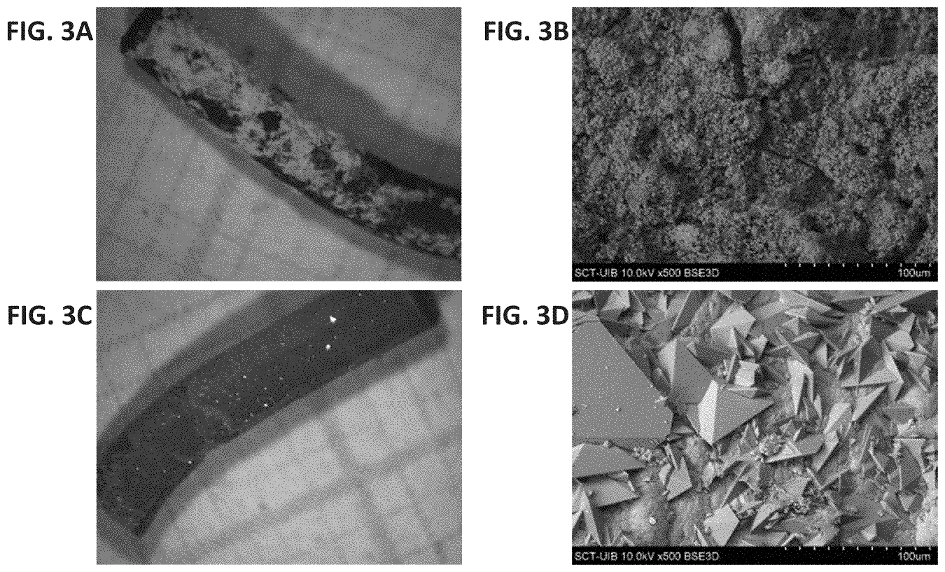

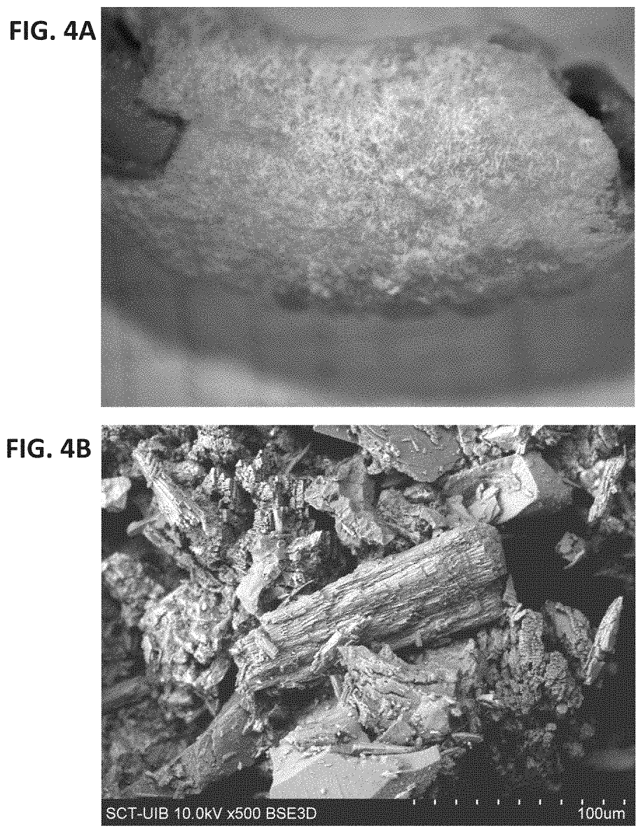

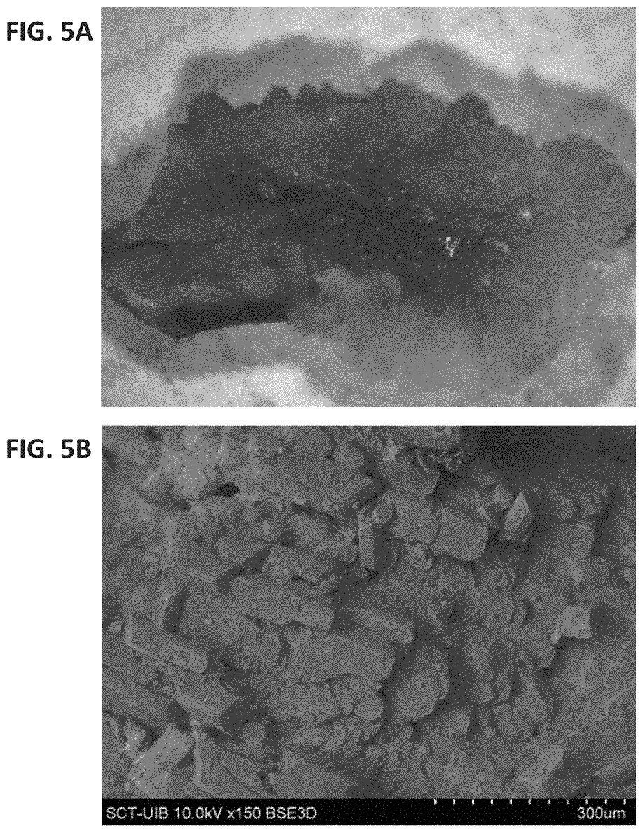

The non-continuous deposits of thickness ranged from 1 to 2 mm, mainly consisted of COD and COM crystals (7.1% in the bladder part and 3.0% in the renal part) and brushite (1.0% in the bladder and renal part), and hydroxyapatite (1.0% in the renal part), as can be seen in FIG. 3A-3D. Large scales (thickness greater than 2 mm), which can cause obstructions, were mainly brushite and hydroxyapatite (3.0% in the renal part and 4.0% in the bladder part, shown in FIGS. 4A and 4B), magnesium ammonium phosphate (2.0% in the bladder part and 1.0% in the renal part, FIGS. 5A and 5B).

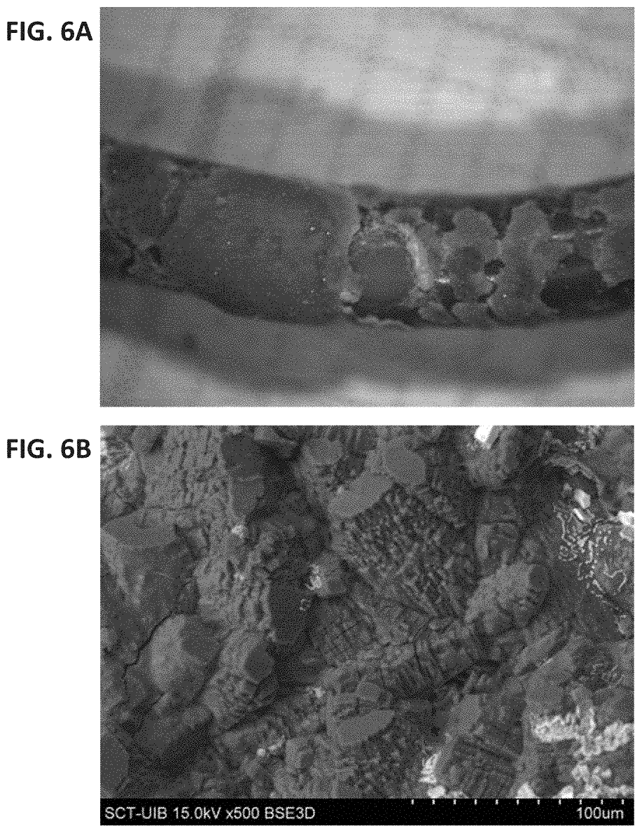

Other important deposits (total coating, thickness less than 2 mm) were hydroxyapatite (1.0% only in the bladder part), uric acid (3.0% in the bladder part and 2.0% in the renal part; FIGS. 6A and 6B) and magnesium ammonium phosphate (1.0% in the renal part). Although the deposits of magnesium ammonium phosphate are clearly of bacterial colonization origin, the presence of bacteria on the crystals was not detected.

TABLE 2
Scale types observed on the stent. Bladder end (location) (N = 99)

| | Magnitude of the scale | | | |
|---|---|---|---|---|
| Type of scale | 1 | 2 | 3 | 4 |
| Organic matter | 12 (12.1%) | | | |
| Calcium oxalate monohydrate | 24 (24.2%) | | | |
| Calcium oxalate monohydrate + calcium | 7 (7.1%) | 7 (7.1%) | | |
| oxalate dihydrate | | | | |
| Brushite | 1 (1.0%) | 1 (1.0%) | | 1 (1.0%) |
| Hydroxyapatite | 1 (1.0%) | | 1 (1.1%) | |
| Hydroxyapatite + brushite | 1 (1.0%) | | | 3 (3.0%) |
| Uric acid | | | 3 (3.0%) | |
| Uric acid + calcium oxalate monohydrate | 1 (1.0%) | | | |
| Bacteria | 4 (4.0%) | | | |
| Hydroxyapatite + ammonium magnesium | | | | 2 (2.0%) |
| phosphate | | | | |
| Brushite + calcium oxalate dihydrate | 1 (1.0%) | | | |
| Ammonium urate | 1 (1.0%) | | | |

TABLE 3
Scale types observed on the stent. Renal end (location) (N = 99)

| | Magnitude of the scale | | | |
|---|---|---|---|---|
| Type of scale | 1 | 2 | 3 | 4 |
| Organic matter | 8 (8.1%) | | | |
| Calcium oxalate monohydrate | 26 (26.3%) | 1 (1.0%) | | |
| Calcium oxalate monohydrate + calcium | 8 (8.1%) | 3 (3.0%) | | |
| oxalate dihydrate | | | | |
| Brushite | 1 (1.0%) | 1 (1.0%) | | |
| Hydroxyapatite | | 1 (1.0%) | | |
| Hydroxyapatite + brushite | | | | 3 (3.0%) |
| Uric acid | | | 2 (2.0%) | |
| Uric acid + calcium oxalate monohydrate | 1 (1.0%) | | | |
| Bacteria | 2 (2.0%) | | | |
| Hydroxyapatite + ammonium magnesium | | | 1 (1.0%) | 1 (1.0%) |
| phosphate | | | | |

As can be seen in Table 1, if one compares calcium deposited in stents classified as type 1 with type 4 the amount of calcium found in the latter is of the order of one thousand times higher than that of the former.

Discussion

From the study of the 99 stents included in this clinical trial, it follows that the first material that is deposited on the stent is organic matter that is present in the urine and that generates a first layer of a few microns (FIG. 1). The thickness and composition of this layer will depend on the composition and concentration of the patient's urine.

For a patient with non-lithogenic urine (no hypercalciuria and/or hyperoxaluria, normocitraturia, and urinary pH of 5.5 to 6.0) and no bacterial colonization of the urine, organic matter deposits can occur, and can act as a heterogeneous nucleants that support the growth of COM crystals over 2 to 3 months. This growth is very slow, so there is only a thin layer (thickness of several micrometers) (FIG. 1). The underlying mechanism may be analogous to the formation of COM stones in renal cavities. If a patient has a high level of urinary calcium, then COD crystals may develop.

If bacteria are present, they can colonize the stent surface and grow while embedded in the initially deposited organic matrix (FIG. 2). The biofilm resulting from infection by urease-producing bacteria increases the urinary pH, and leads to the formation of carboxyapatite and magnesium ammonium phosphate crystals (FIG. 5A, FIG. 5B). Depending on bacterial activity, these crystals can range from small deposits to large concretions, and in many cases they obstruct flow through the stent and make stent extraction more difficult. The most common bacteria in these deposits is P. mirabilis.

For urine with a pH greater than 6.0 and no bacterial colonization, significant deposits of calcium phosphate can develop, depending on the specific conditions. In particular, when the urine has a high calcium concentration, a citrate deficit, and a pH greater than 6, large deposits of brushite can develop (FIG. 5A, FIG. 5B). Under these conditions, large COD crystals can also occur. When the calcium and magnesium concentrations are low, large hydroxyapatite deposits can develop. In addition, these different types of deposits can coexist within the same patient. For urine with a pH less than 5.5, major deposits of uric acid can develop (FIG. 6A, FIG. 6B).

As can be seen, the process begins with the deposit of organic matter on the stent. Depending on the presence of bacteria (infection) and the composition of the urine, the different encrustations that have been described can be generated.

Conclusions

The present study examined the development of stent encrustations, a potentially serious clinical problem because the encrustations can obstruct flow through the stent and make stent removal more difficult. There were several major findings. First, it is necessary to determine the presence of urinary infections. An infection by ureolytic bacteria leads to a marked increase in urinary pH and generation of ammonium. Under these conditions, crystalline deposits of carboxyapatite and magnesium ammonium phosphate develop; in addition to the complications generated by the infection itself, these deposits can make stent removal more difficult. Second, in the absence of infection but in the presence of a urinary pH persistently above 6.0 and other lithogenic factors (high calcium concentration and a deficiency of lithogenic inhibitors), deposits of brushite or brushite+hydroxyapatite can develop. Third, in the absence of infection but in the presence of a urinary pH persistently lower than 5.5, major deposits of uric acid can develop on a stent. In all cases the development of scales on the stent begins with a deposit of organic matter.

### Example 2

### Reduction of Urinary Tract Stent Encrustation by Nutraceutical Intervention on Urine pH

The main goal of this study was to assess the efficacy and safety of a nutraceutical on the prevention of double-J stent encrustation. Secondary objectives included urine pH decrease, stent removal, incidence of adverse events, patient's compliance and physician's and patient's satisfaction.

Material and Methods

1. Study design: This prospective, parallel, double-blinded, randomized and placebo-controlled trial was conducted at 9 public hospitals from Spain, in accordance with the Declaration of Helsinki, ethical standards, current legislation and GCPs. The study was approved by local Ethics Committees, and informed consent was obtained from all patients prior to their enrolment in the study.

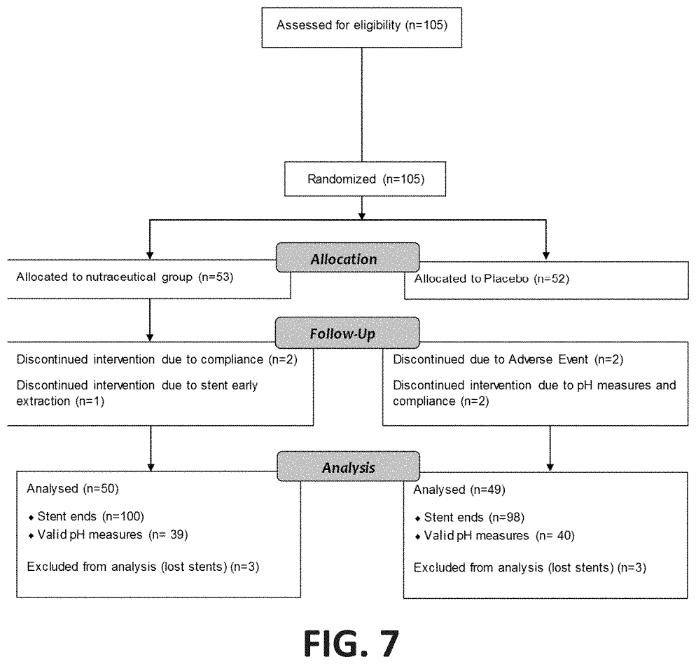

2. Subjects: Inclusion criteria comprised patients aged 18 and older, capable of daily self-monitoring of their urine pH, who were recently implanted with a double J stent (less than a week) or were about to get it implanted (FIG. 7). Exclusion criteria comprised patients with programmed stent removal before 3 weeks from inclusion visit, those with underlying pathologies that could be incompatible with the nutraceutical consumption, and uric or cystinuric patients in which different pH control recommendations are needed.

3. Treatment description: Subjects were randomly assigned in a 1:1 ratio to receive a urine acidifier nutraceutical or placebo as investigators included them in a password-protected computer database with a pre-programmed randomization list with blocks of 2 to 4. The nutraceutical arm consisted in oral administration of three capsules per day (1-1-1) of the LIT-CONTROL DEVICARE® pH Down dietary supplement (each capsule contains: L-methionine, rice bran extract enriched with calcium magnesium phytate, zinc gluconate and vitamin A; Table 4) to maintain the urine pH under 6.2, a preventive pH value to avoid stent encrustation. Patients in placebo arm received a placebo treatment consisting in oral capsules with the same organoleptic and posology characteristics as the investigational compound. Both arms used a device to self-monitor their urinary pH every morning, and identical hygienic-dietary indications for stent care were given to all participants.

TABLE 4
Composition of LIT-CONTROL DEVICARE ® pH Down dietary
supplement

| Ingredient | mg/capsule |
|---|---|
| L-methionine | 500 |
| Rice bran extract (calcium magnesium phytate) | 170 |
| Zinc gluconate | 5 |
| Vitamin A | 0.15 |
| Maltodextrin | 4.85 |
| Sipernat 50S (Silicium dioxide) | 25 |
| Magnesium stearate | 10 |

4. Follow-up evaluation: Intervention and pH self-control duration ranged from 3 to 8 weeks depending on the time-lapses between the baseline visit and the stent removal. Once removed, the process consisted in submerging the stent ends in thymol to cleanse, gently letting them air dry over paper. All analyses were carried out in a central laboratory. Stents were cut and processed to examine the renal and vesical ends separately.

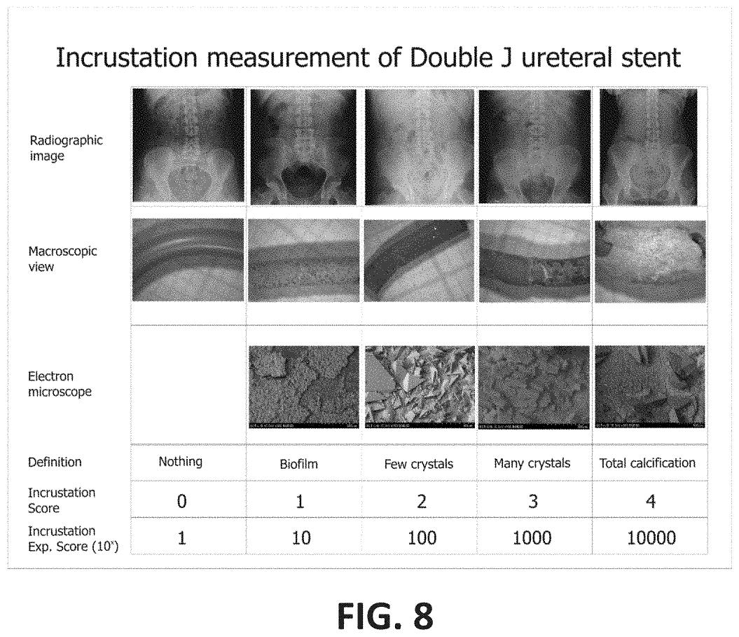

5. Outcome measures: Double J stent ends were separately analysed to determine the presence and degree of encrustation and data were arranged in two databases one for patients and the other for stent ends. The presence and degree of stent encrustation were the primary outcome measures to test the efficacy of the nutraceutical. The degree of calcification was evaluated using a qualitative 5-level score (0: nothing; 1: biofilm; 2: few crystals; 3: many crystals; 4: global calcification) (FIG. 8). After analysing calcium concentrations of two samples of score 1 and score 4 stent ends by Arsenazo III spectrophotometry an exponential transformation (0-10000) of the encrustation variables was done since calcium concentration ratio between stents with 4 score and stents with 1 score ranged 1:600 to 1:1500. A dichotomous variable for global calcification (score 4) was created and used in the stent ends database. The type and size of crystals were assessed by SEM and micro-analysis by dispersive energy of XR, and the degree of global calcification of each end was measured using ICP-AES spectroscopy (FIG. 8). As secondary outcome measures, urine pH values from 24 h urine analysis were considered day 0 pH values, mean values of urine pH from days 1-3 were considered the baseline of pH self-monitoring data and were compared to pH values at day 21 and to mean pH values from day 4 to the end of indwelling period (21 to 56 days). Duration and method of the stent extraction intervention data were also recorded as secondary outcomes. Risk factors for encrustation development (stent material, days with implanted stent and number of previous implantations), stent-related symptoms, previous uropathies and sociodemographic data were also recorded to be studied as factors or covariates. Compliance was measured by counting returned medication and consumption of more than 80% of the capsules was considered good adherence.

6. Statistical analysis: A sample size of 47 evaluable patients per treatment group would provide approximately 80% power to detect a reduction with an effect size of 0.6 in the calcification score in either nutraceutical group versus placebo using a Mann-Whitney U test. The sample was increased to 105 participants considering a 10% loss to follow up. Demographic and baseline characteristics and safety and tolerability data were summarized using descriptive statistics. The primary endpoint, the difference in the calcification score between groups was assessed using a Fisher exact test for the categorical variable global calcification and a Mann-Whitney U test for the calcification scores, which were also analysed using Generalized Linear Models for a Tweedie distribution with a logarithmic link, including treatment, sex, baseline pH<6 and indwelling duration >39 days as fixed factors and age as a covariate. Mean differences and 95% confidence limits were calculated for all comparisons between groups. Global calcification was analysed using a logistic regression model that included treatment, sex, baseline pH<6, first implantation, stent material, indwelling duration and age. Secondary end points as pH reduction, intervention time for stent removal or patient satisfaction were analyzed using one-way analysis of variance. Statistics for all tables, figures, and graphs were calculated from the total number of valid cases. All statistical analyses were performed on the intention-to-treat population using SPSS 22.0 software for Windows.

Results

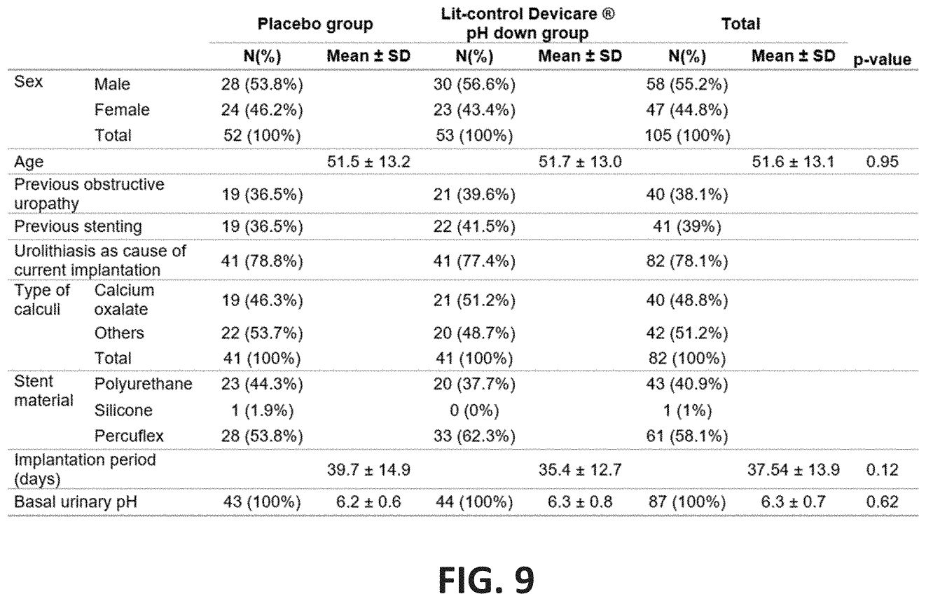

A total of 105 patients with a mean (SD) age of 51.6 (13.1) years, and 55.2% male were analysed (FIG. 7 and FIG. 9). A number of 198 stent ends were collected from 99 subjects who wore them for an average time-span of 37.54±13.9 days.

1. Global calcification of all stent ends: Eight stent ends (8.2%) showed global calcification in the placebo group and 1 (1.0%) in the nutraceutical group (R.R.: 8.2 [1.04-64.06]; p=0.018).

2. Calcification score of all stent ends: Regarding the degree of the deposits, the analysis of all the double J stent ends resulted in calcification levels of 847.9 (2746.3) in the placebo group, and of 187.6 (1023.0) in the nutraceutical group (p=0.021); difference (95% IC) is 660.4 (81.69, 1239.0).

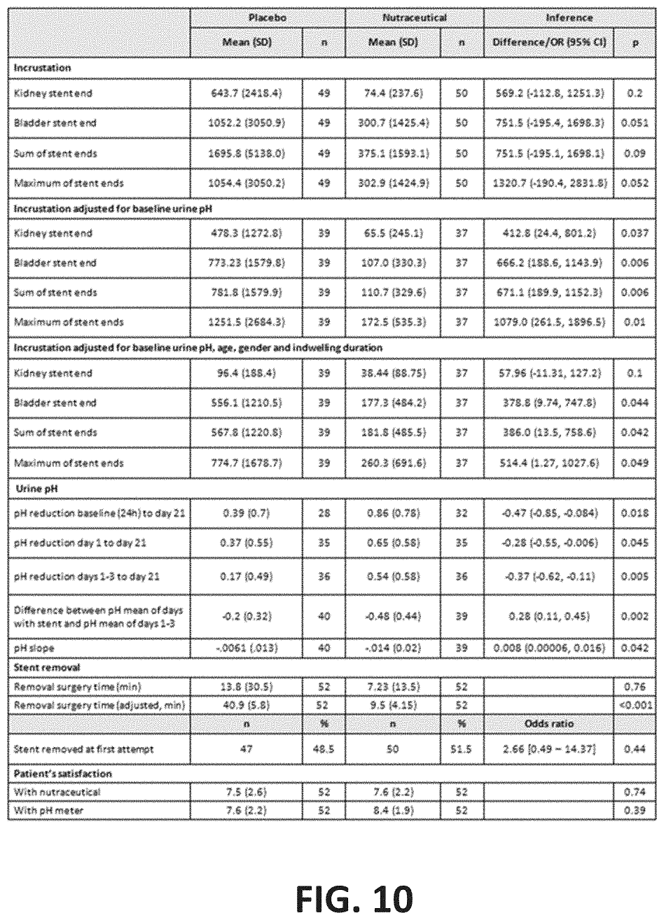

3. Calcification scores by stent end: Calcification scores by stent end are detailed in FIG. 10.

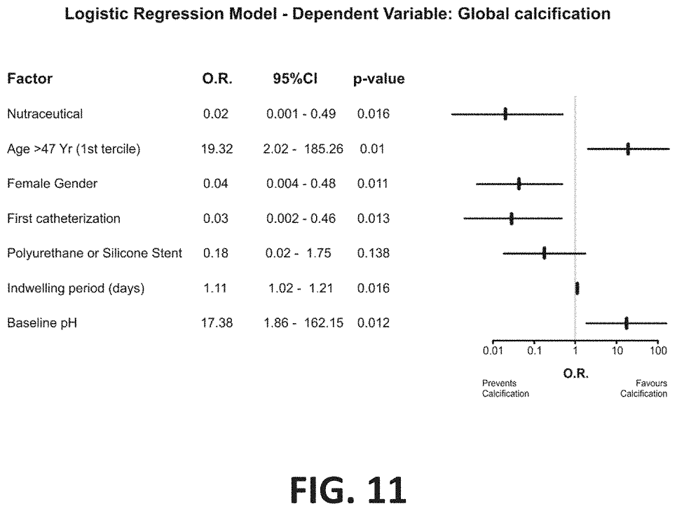

4. Multivariate model of global calcification: Binary logistic regression model of all stent ends global calcification showed a O.R. in the placebo group of 49.34 [95% IC: 2.04-1193,25; p=0.016] emerging as protective factors age >47, first implantation, polyurethane or silicone stents and baseline pH<6 and favoring calcification would be male gender (FIG. 11).

5. pH reduction: A secondary objective was to assess the efficacy of the nutraceutical in the reduction of the pH level. Differences between baseline pH to values registered during implantation were significantly greater in the nutraceutical group (FIG. 10).

6. pH during indwelling and calcification: Four (22%) of 18 patients whose mean pH level during indwelling was greater than 6 showed global calcification in 7 stent ends and 1 (1.7%) of 58 patients with lower pH levels showed this outcome in 1 stent end (RR: 12.9 [1.4-296.7]; p<0.012).

7. Duration of implantation and calcification score: Spearman correlation between indwelling time in days and encrustation score was p=0.345 (p<0.001) for the kidney end and p=0.211 (p<0.037) for the bladder end. When separated by study group, r^{2 of encrustation score at kidney end by indwelling time was} 0.079 for the placebo group and 0.018 for the nutraceutical group.

8. Compliance: Fifteen patients (37.5%) in the placebo group and 12 (30%) in the nutraceutical group took less than 80% of prescribed doses (p=0.6). Twelve patients (11.4%) in the placebo group and 14 (13.3%) in the nutraceutical group failed to provide valid pH measures due to problems with the device.

9. Tolerability and security: Three patients in the placebo group reported mild adverse events (2 nausea and 1 hot flashes) and 3 in the nutraceutical group (1 diarrhea, 1 blurry vision and 1 dyspepsia). Two patients in the placebo group discontinued the study due to adverse events. No other measures were taken for the rest of patients with adverse events. Six patients in the placebo group and 6 patients in the nutraceutical group were prescribed with antibiotics due to positive baseline urine cultures.

Discussion

The calcification phenomenon has relevant clinical consequences that may compromise stent removal. When indwelling time increases, encrustation prevalence increases proportionally and global calcification can occur, leading to the use of endourology techniques, extracorporeal lithotripsy or open surgery to resolve these conditions. Although heavily encrusted stents clearly do pose significant problems, minor encrustations can also challenge the endourologist, particularly if occurring frequently and repetitively. Among 105 participants in this study only 9 stent ends showed global calcification but the difference between nutraceutical group to placebo yielded a relative risk of 8.2 and this effect was enhanced when adjusted by age, sex, previous implantation, stent composition, indwelling duration and baseline pH level.

The level of deposits was significantly lower in the nutraceutical group with an effect size (Cohen's d) of 0.32, which is lower than the one estimated in the sample size calculation (d=0.6). However, it increased when calcification was adjusted for other factors. Furthermore, effect size of global calcification as a dichotomous variable yielded a RR of 8.2 which is equivalent to a Cohen's d greater than 1.

The multivariate models showed that the formation of deposits in the double J stent ends is a multifactorial process dependent of patient's previous implantation, stent composition, duration of the implantation period, baseline pH level, and the use of a nutraceutical.

A relationship between baseline pH and calcification has been observed in the multivariate analysis showing a pH lower than 6 acts as an independent factor against high calcification scores. Furthermore, a mean pH greater than 6 during indwelling increased 12.9 times the risk of global calcification of a stent end. Since 26.6% of patients (23% in the nutraceutical group and 30% in the placebo group) had a mean pH greater than 6 during indwelling, it can be stated that a not negligible proportion of patients with a double J stent are at risk of global calcification due to their urine pH.

Both nutraceutical and placebo groups lowered their urine pH levels from baseline to day 21 or against the average pH levels (day 4 to end of indwelling period) and also placebo and nutraceutical groups pH slopes, though they significantly differ, both they showed a decreasing direction. This may be due to the fact that hygienic-dietary indications for stent care were given to all participants but also and specially to the daily urine pH self-monitoring carried out by both two groups.

Eight patients in the placebo group and 10 in the nutraceutical group failed to provide valid pH measures due to systematic calibration errors, nevertheless patients scored their satisfaction with the device with an average of 8 over a 0 to 10 scale.

The control of the urinary pH level using a nutraceutical and urinary pH self-monitoring using a device at home was more efficacious than the use of a placebo plus pH self-monitoring in reducing double J ureteral stent calcification and lowering urine pH levels.

The duration of the stent extraction procedure, when adjusted by previous implantations and symptoms associated with the presence of the stent, showed significant differences on the marginal means between the placebo and treatment groups.

Adjusted analyses showed that both nutraceutical and baseline pH are independent factors that prevent stent calcification. The fact that the nutraceutical studied consists in an acidifier (L-methionine) plus a calcification inhibitor (phytate) may explain it, since both components have a synergic effect on reducing urinary pH which may prevent crystallization processes due to organic film growing.

Conclusion

The use of a nutraceutical in patients indwelling a double J ureteral stent resulted in fewer calcifications, making the removal process easier for the urologist and safer for the patient.

We can conclude that the nutraceutical reduces more than 8 folds the probability of one of the double J stent ends global calcification.

### Example 3

### Analysis of Clinical Trial Results

Assessment of Double J Stents Encrustation, Statistical Analysis Methodology

The numbers corresponding to the score in encrustation/calcification scale were transformed in exponentials in base 10 before analysis (10^{x=encrustation}). This is due to the asymptotic behavior of the crystals accumulation ranging from a microscopic presence until a great macroscopic one, which prevents the stent removal with the usual procedure.

This encrustation/calcification measurement was assessed for each stent end (kidney and bladder) and new variables were generated; one corresponding to the sum of the encrustation in both ends and another to the score in the end with the highest encrustation level.

Considering that encrustation starts in one stent end and that this process may be independent from the other stent end, a database was generated for the cases where encrustation corresponded to the stent ends (n=198) to allow the analysis of encrustation starting in all possible spots in the stent.

Presence (Yes/No) and calcification degree (0-IV) were at the researcher discretion.

Results are shown in FIG. 8.

Case Distribution According to Encrustation Levels: Kidney Vs. Bladder

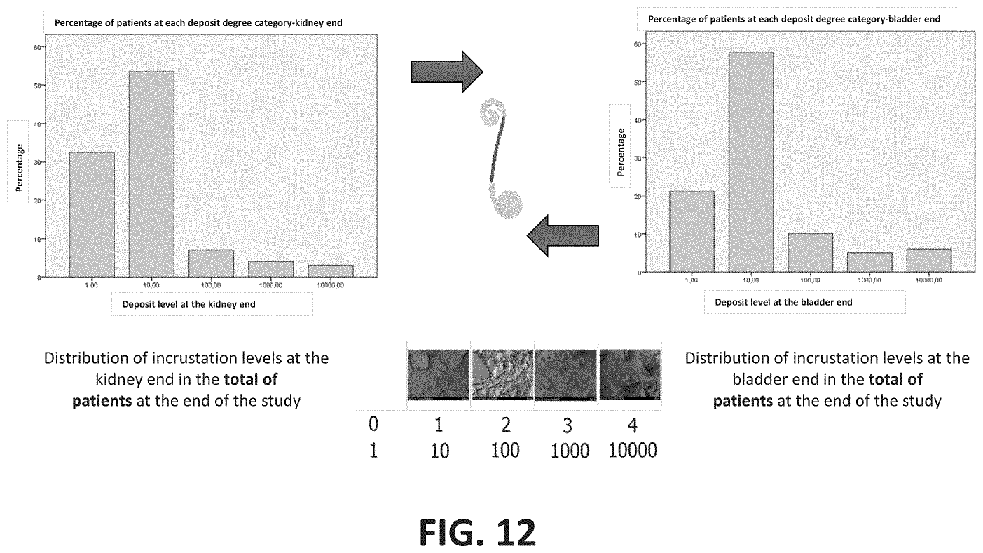

FIG. 12 shows the distribution of encrustation levels at the kidney end and at the bladder end in the total of patients (placebo and treatment group) at the end of the study. It can be seen that the most widely prevalent situation is the generation of organic film as a first stage of encrustation (scale magnitude 1). It can be seen that the situation in the bladder end was worse than in the kidney end.

Main Objective: Encrustation Prevention. Conclusion 1

After analyzing all stent ends (n=198), in the placebo group there were 8 global calcifications (grade 4) and only 1 in the experimental LIT-CONTROL DEVICARE® pH Down (O.R: 8.8). This effect increased when multivariate tests were performed. The multivariate tests were adjusted according to age, sex, previous stent implantation, stent composition, duration of implantation and baseline pH at days 1-3.

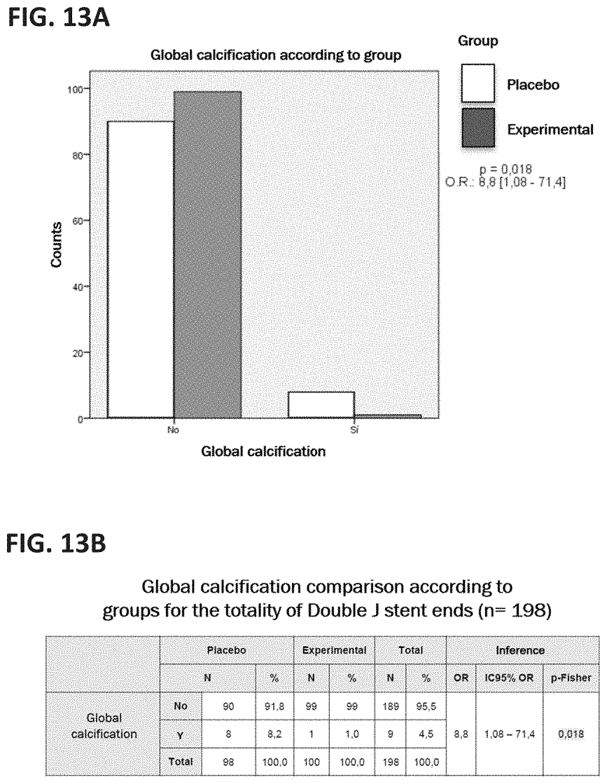

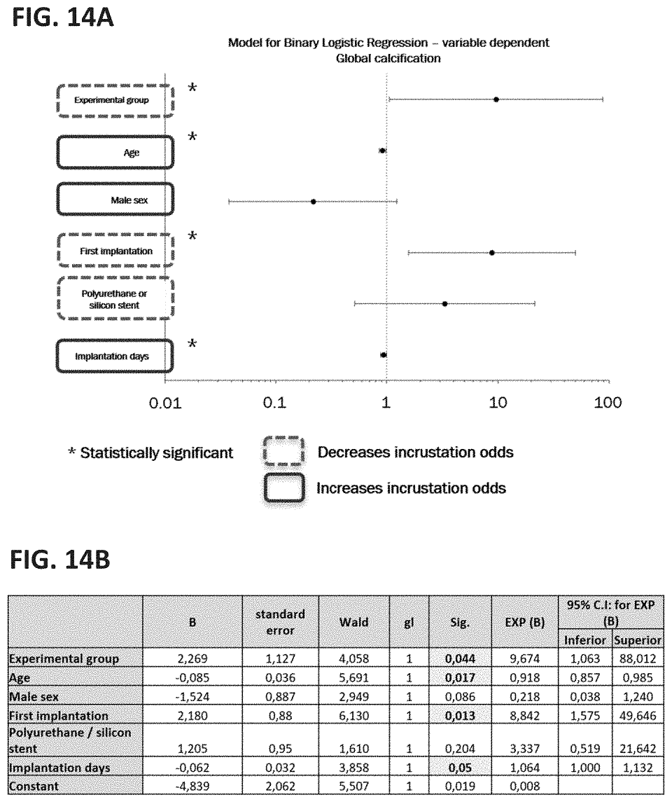

Results are shown in FIG. 13A, FIG. 13B, FIG. 14A, FIG. 14B. It was observed a statistically significant difference in the number of stent ends affected by global calcification, i. e., with a score=4 (or 10.000 in log scale). p=0.018. The obtained OD gives a 8-fold lower probability for encrustation when placed in the experimental group.

One can conclude that LIT-CONTROL DEVICARE® pH Down decreases more than 8-fold the probability that one stent end will reach global calcification.

Main Objective: Encrustation Prevention. Conclusion 2

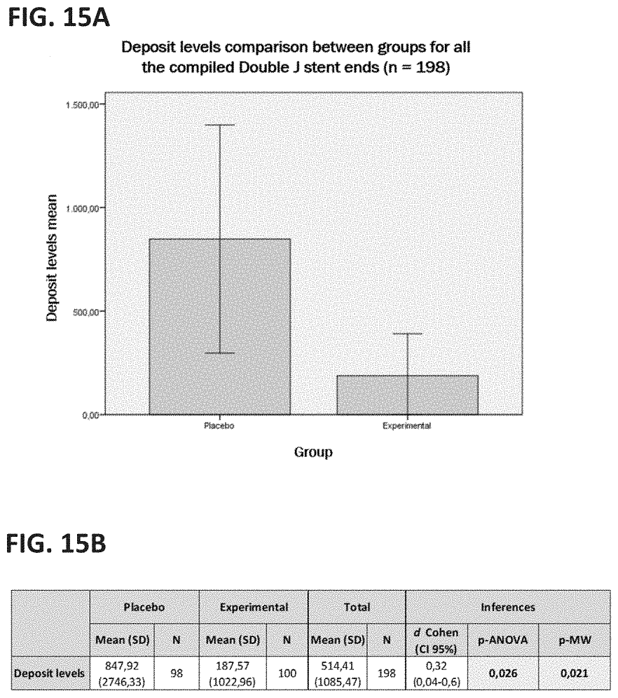

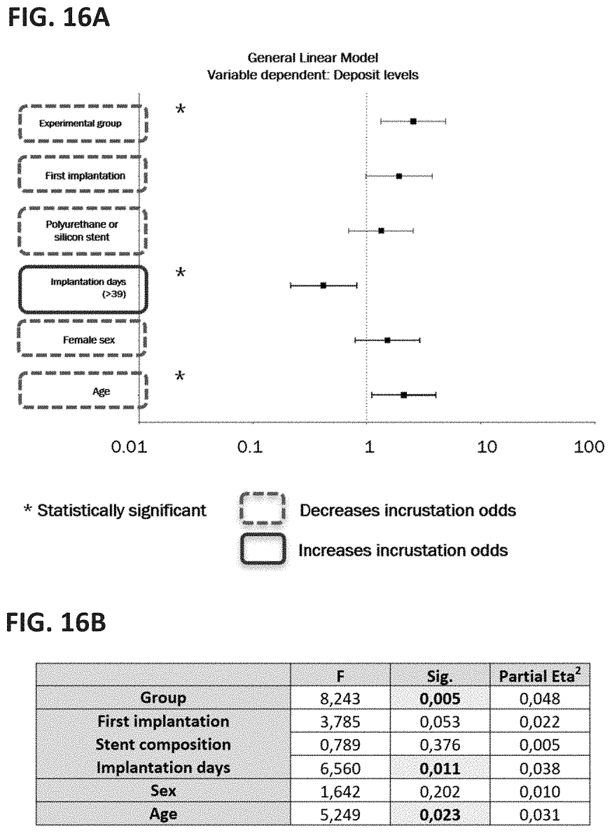

Concerning the deposit levels, one can observe in the analysis for all stent ends a calcification level of 847.92 (2746.33) in the placebo group, while the LIT-CONTROL DEVICARE® pH Down group presented levels like 187.57 (1022.96), with a p=0.02, and with a Cohen size effect=0.32 ([0.04-0.06], which may increase when adjusted by other factors. Results are shown in FIG. 15A, FIG. 15B, FIG. 16A and FIG. 16B. It was observed a statistically significant difference in the encrustation levels considering all stent ends for the treatment groups (p=0.026). This means considering all encrustation values, for both renal and bladder ends.

Thereby, the proposed experimental treatment reduced significantly the deposit levels in Double J stent ends.

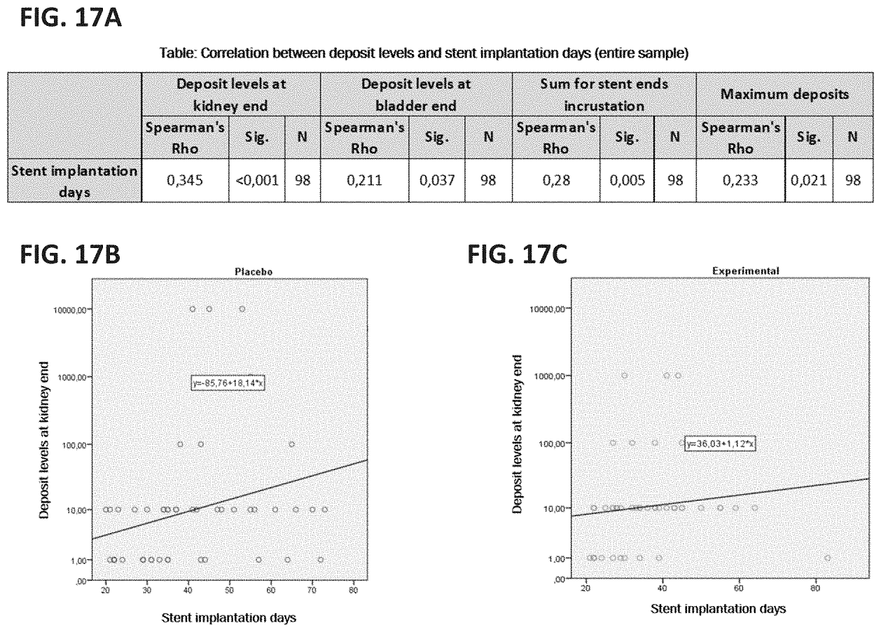

FIG. 17A-FIG. 17C show the relation between encrustation and days of stent implantation in the experimental and placebo groups. Graphs and projections show that the resultant slope counterposing encrustation levels and stent implantation days is less pronounced in the experimental group when compared to placebo. One can conclude that LIT-CONTROL DEVICARE® pH Down confers a protection from encrustation associated with the number of days with a stent indwelled.

Main Objective: Encrustation Prevention. Conclusion 3

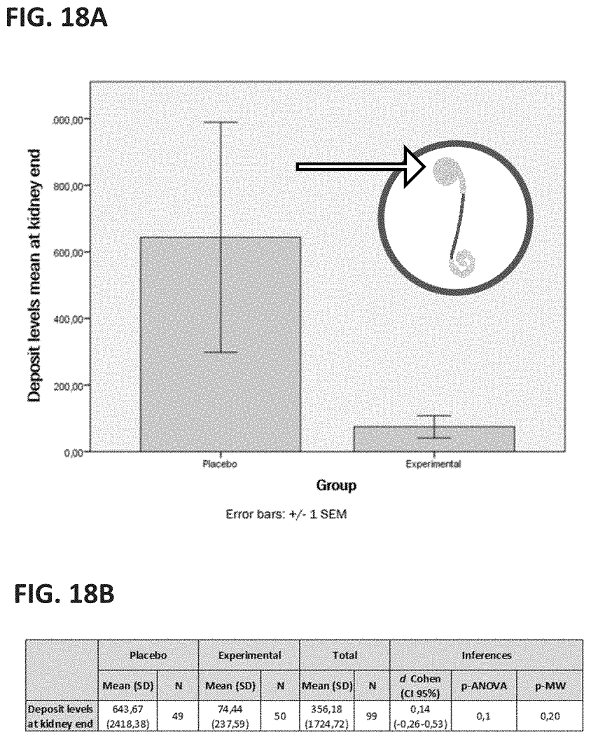

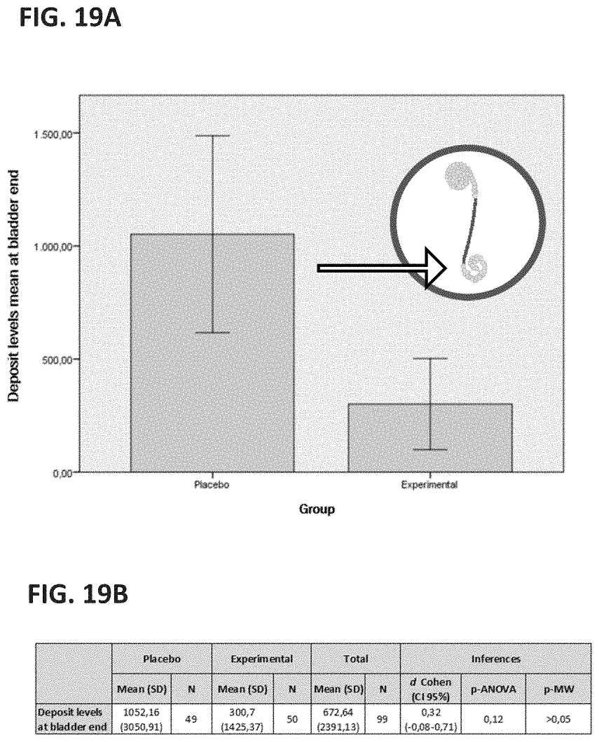

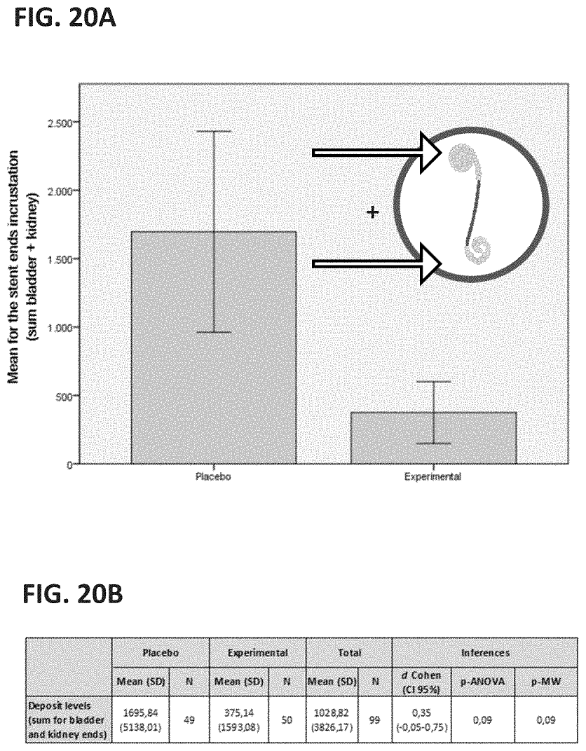

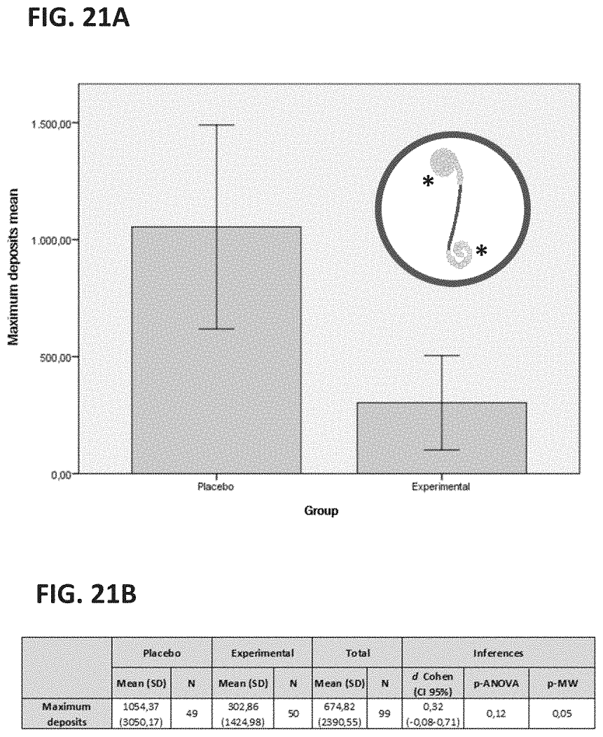

Results are shown in FIG. 18-21. In the encrustation analysis weighing (a) deposits at kidney end, (b) deposits at bladder end, one can systematically observe a reduced encrustation in the experimental group treated with LIT-CONTROL DEVICARE® pH Down when compared to placebo. Even though not statistically significant, a tendency is demonstrated with p values ranging from 0.05 to 0.2. The d Cohen size effect=0.14 [−0.26-0.53] a 0.35 [−0.05-0.75].

Main Objective: Encrustation Prevention. Conclusion 4

Results are shown in FIG. 18-21. The encrustation values for (a) deposits detected at the kidney end (b) deposits detected at the bladder end, (c) sum for deposits in both stent ends, (d) maximum deposits at both stent ends, were submitted to multivariate analysis as General Lineal Models in order to adjust the results to population variables as (1) age, (2) sex, and other variables related to encrustation as (3) implantation days, (4) previous implantation, (5) stent composition and (6) baseline pH at days 1-3.

The combination of the 4 variables encrustation-dependent (a-d) with the 6 added factors (1-6) generated 60 General Lineal Models from which 36 were statistically significant for the contribution of LIT-CONTROL DEVICARE® pH Down for a drastic reduction in stent encrustation. The remaining Models showed a strong tendency for the statistical significance.

Main Objective: Encrustation Prevention. Global Conclusion

The deposition in Double J stent ends seems to be a multifactorial process where the urinary pH acidification and the increase in inhibitory substances due to LIT-CONTROL DEVICARE® pH Down may confer a prevention.

Secondary Objective: pH Control. Conclusion 1

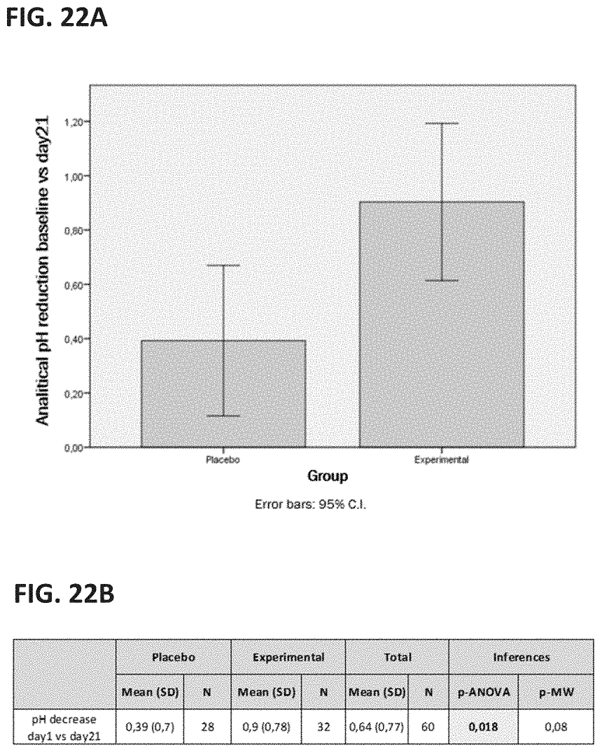

Results are shown in FIG. 22A and FIG. 22B. Concerning the pH values at day 21 vs day 1/baseline, the placebo group reached a 0.37 (0.55) decrease in pH degrees versus a 0.65 (0.58) decrease in the experimental group LIT-CONTROL DEVICARE® pH Down (p=0.018; d de Cohen=0.33 [0.0-0.81].

Secondary Objective: pH Control. Conclusion 2

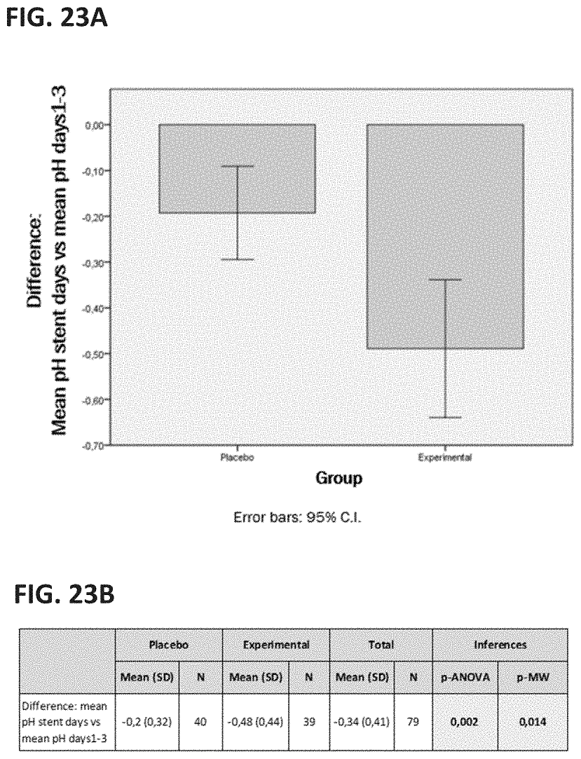

Results are shown in FIG. 23A and FIG. 23B. For the difference between the mean pH values during stent implantation and the mean pH values for days 1-3, the placebo group reached a decrease of −0.2 (0.32) pH degrees versus a decrease of −0.48 (0.44) in the LIT-CONTROL DEVICARE® pH Down group (p=0.002; Cohen d=0.85 [0.39-1.31]

Secondary Objective: pH Control. Conclusion 3

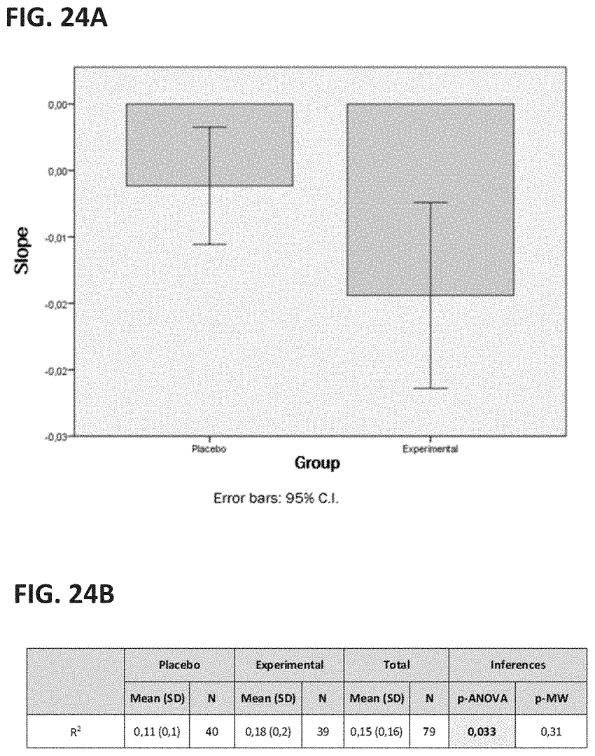

Results are shown in FIG. 24A and FIG. 24B. Concerning the difference in the regression slope for the pH values along the stent implantation days, the placebo group revealed a r² value of 0.11 (0.1) versus a value of 0.18 (0.2) observed for the LIT-CONTROL DEVICARE® pH Down group (p=0.033; Cohen d=0.63 [0.18-1.09].

Secondary Objective: pH Control. Global Conclusion.

In addition, and globally, it was demonstrated a statistically significant relation between the accomplished pH reduction during stent implantation days and the levels of deposits detected after stent removal.

Secondary Objective: Cost-Effectivity. Conclusion 1

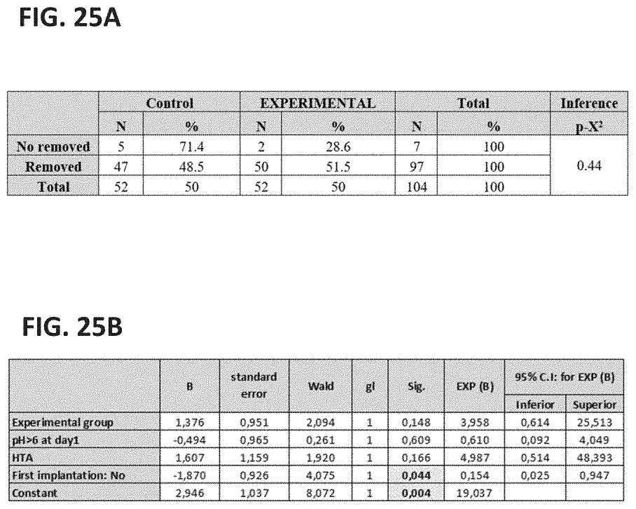

Results are shown in FIG. 25A and FIG. 25B. During this study, 7 cases of impossibility of stent removal in the first trial were registered, 5 cases in the placebo group and 2 cases in the LIT-CONTROL DEVICARE® pH Down group (p=0.44) which, after an adjustment for previous implantation, pH>6 at day 1/baseline and hypertension, showed a favorable tendency (O.R.: 3.9 [0.6-25.5], p=0.148), even though not reaching statistical significance.

Secondary Objective: Cost-Effectivity. Conclusion 2

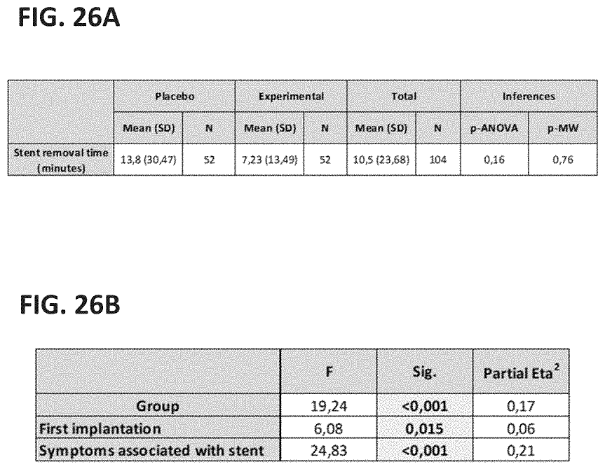

Results are shown in FIG. 26A and FIG. 26B. The duration time for the stent removal was of 13.8 (30.47) minutes for the placebo group and 7.23 (13.49) minutes for the LIT-CONTROL DEVICARE® pH Down group (p=0.16).

When adjusting these values to factors as previous implantation and symptoms associated with the presence of the stent, the marginal measurements were 40.9 [29.3-52.4] minutes for the placebo group and 9.5 [1.3-17.7] minutes for the LIT-CONTROL DEVICARE® pH Down group.

Secondary Objective: Safety. Conclusion 1

LIT-CONTROL DEVICARE® pH Down was tolerated as well as the placebo treatment, registering 3 adverse reactions for the placebo group and 3 for the experimental group. None of these were severe, and in no circumstance was demonstrated or discarded a correlation between the adverse events and the concerned product. Two patients abandoned the treatment in the placebo group.

Concerning the adherence/compliance to the treatment, a monitoring was performed with a recounting of the returned medication after the trial period. It was observed that 71% of the patients had taken more than 80% of the prescribed doses. There was no negative correlation between adherence and efficacy.

### Example 4

### Combined Use of Urinary pH Control Compositions

1. Subjects: Inclusion criteria comprise patients aged 18 and older, capable of daily self-monitor their urine pH, who have been recently implanted with a double J stent (less than a week) or are about to get it implanted. Exclusion criteria comprise patients with programmed stent removal before 3 weeks from inclusion visit, those with underlying pathologies that could be incompatible with the nutraceutical consumption, and uric or cystinuric patients in which different pH control recommendations are needed.

2. Treatment description: Subjects are randomly assigned in a 1:1 ratio to receive a nutraceutical or placebo as investigators include them in a password-protected computer database with a pre-programmed randomization list with blocks of 2 to 4. Each patient in the nutraceutical arm is provided with three oral compositions to be self-administered depending on the pH value reported by the pH meter. The three oral compositions are the following:

LIT-CONTROL DEVICARE® pH Down dietary supplement (each capsule contains: L-methionine, rice bran extract enriched with calcium magnesium phytate, zinc gluconate and vitamin A; Table 4) to maintain the urine pH under 6.2, a preventive pH value to avoid stent encrustation.

LIT-CONTROL DEVICARE® pH Up dietary supplement (each capsule contains: potassium citrate, magnesium citrate, ginseng extract rich in saponins (30%), lemon juice powder, and zinc gluconate; Table 5) to maintain the urine pH over 5.5, a preventive pH value to avoid stent encrustation.

TABLE 5
Composition of LIT-CONTROL DEVICARE ® pH Up dietary supplement

| Ingredient | mg/capsule |
|---|---|
| Potassium citrate | 200 |
| Magnesium citrate | 200 |
| Ginseng extract rich in saponins (30%) | 150 |
| Lemon juice powder | 120 |
| Zinc gluconate | 20 |
| Maltodextrin | 20 |
| Sipernat 50S (Silicium dioxide) | 30 |
| Magnesium stearate | 10 |

LIT-CONTROL DEVICARE® pH Balance dietary supplement (each capsule contains: rice bran extract enriched with calcium magnesium phytate, magnesium oxide, grape seed polyphenol extract, zinc gluconate and vitamin A; Table 6) to maintain the urine pH between 5.5 and 6.2, a preventive pH value to avoid stent encrustation.

TABLE 6
Composition of LIT-CONTROL DEVICARE ® pH Balance dietary
supplement

| Ingredient | mg/capsule |
|---|---|
| Rice bran extract (calcium magnesium phytate) | 300 |
| Magnesium oxide | 125 |
| Grape seed polyphenol extract | 125 |
| Vitamin A | 0.15 |
| Zinc gluconate | 5 |
| Sipernat 50S (Silicium dioxide) | 5 |
| Magnesium stearate | 10 |

Patients in placebo arm receive a placebo treatment consisting in three oral compositions in form of capsules with the same organoleptic and posology characteristics as the investigational compound. Both arms use a device to self-monitor their urinary pH every morning, and identical hygienic-dietary indications for stent care are given to all participants.

In response to the pH measurement, patients take one of the three compositions: if pH is below 5.5, they take LIT-CONTROL DEVICARE® pH Up; if pH is over 6.2, they take LIT-CONTROL DEVICARE® pH Down, and if pH is within range 5.5-6.2, they take LIT-CONTROL DEVICARE® pH Balance.

Intervention and pH self-control duration range from 3 to 8 weeks depending on the time-lapses between the baseline visit and the stent removal.

3. Outcome measures, quantification and analysis: Double J stent ends are separately analysed to determine the presence and degree of encrustation. Analysis and calculations are performed as in Example 2. It is expected to encrustation will be reduced by keeping the pH levels within the optimal range.

What is claimed is:

1. A method to facilitate the removal of a urinary tract device in a subject in need thereof comprising administering to the subject:
(i) at least one dose of an oral composition that decreases urine pH, if the subject's urine pH is above a pH threshold or pH range;
(ii) at least one dose of an oral composition that increases urine pH, if the subject's urine pH is below a pH threshold or pH range;
(iii) at least one dose of an oral composition that maintains urine pH, if the subject's urine pH is within a pH range; or,
(iv) a combination thereof;
wherein the administration of the compositions to the subject reduces encrustation on the surface of the urinary tract device, and the reduced encrustation facilitates the removal of the urinary tract device.

2. A method to prevent encrustation on a urinary tract device in a subject in need thereof comprising administering to the subject:
(i) at least one dose of an oral composition that decreases urine pH, if the subject's urine pH is above a pH threshold or pH range;
(ii) at least one dose of an oral composition that increases urine pH, if the subject's urine pH is below a pH threshold or pH range;
(iii) at least one dose of an oral composition that maintains urine pH, if the subject's urine pH is within a pH range; or,
(iv) a combination thereof;
wherein the administration of the compositions to the subject reduces encrustation on the surface of the urinary tract device.

3. The method of claim 2, wherein the oral composition that decreases urine pH comprises a urinary acidifying agent and a calcium phosphate crystallization inhibitor.

4. The method of claim 3, wherein the urinary acidifying agent is an amino acid or a combination thereof.

5. The method of claim 4, wherein the amino acid is L-methionine.

6. The method of claim 3, wherein the calcium phosphate crystallization inhibitor comprises phytic acid, a salt thereof, or a combination thereof.

7. The method of claim 2, wherein the oral composition that increases urine pH comprises a urinary basifying agent and a uric acid crystallization inhibitor.

8. The method of claim 7, wherein the urinary basifying agent comprises citric acid, salts thereof, or combinations thereof.

9. The method of claim 7, wherein the uric acid crystallization inhibitor comprises a saponin.

10. The method of claim 2, wherein the oral composition that maintains urine pH comprises (i) a calcium phosphate crystallization inhibitor, and (ii) a magnesium salt, magnesium hydroxide, magnesium oxide, or a combination thereof.

11. The method of claim 10, wherein the calcium phosphate crystallization inhibitor comprises phytic acid, a salt thereof, or a combination thereof.

12. The method of claim 10, wherein the oral composition that maintains urine pH comprises magnesium oxide.

13. The method of claim 10, wherein the oral composition that maintains urine pH further comprises at least one polyphenol.

14. The method of claim 2, wherein the urinary tract device is a stent.

15. The method of claim 14, wherein the stent is a JJ stent.

16. The method of claim 2, wherein the administration of the compositions to the subject decreases encrustation on the surface of the urinary tract device by at least 2-fold, at least 3-fold, at least 4-fold, at least 5-fold, at least 6-fold, at least 7-fold, or at least 8-fold compared to the encrustation observed in control subjects.

17. The method of claim 2, wherein the method further comprises the measurement of urine pH by the subject using a self-diagnostic pH measuring device.

18. The method of claim 2, wherein the oral composition that decreases urine pH comprises L-methionine, rice husk extract enriched in phytic acid magnesium and calcium salts, zinc gluconate, and vitamin A.

19. The method of claim 2, wherein the oral composition that increases urine pH comprises potassium citrate, magnesium citrate, a saponin, zinc gluconate, and vitamin A.

20. The method of claim 2, wherein the composition that maintains urine pH comprises rice husk extract enriched in phytic acid magnesium and calcium salts, magnesium oxide, grape seed extract enriched in polyphenols, zinc gluconate, and vitamin A.
